SEQUENCE LISTING (46 sequences)

SEQ ID NO: 1 - protein, 134 aa, Homo sapiens
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGVLNFGNEKLTFGTGTRLTIIP

SEQ ID NO: 2 - protein, 131 aa, Homo sapiens
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSEDTAYYGYTFGSGTRLTVVE

SEQ ID NO: 3 - protein, 136 aa, Mus musculus
IQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 4 - protein, 173 aa, Mus musculus
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 5 - protein, 22 aa, Artificial Sequence
  Synthetic P2A linker
GSGATNFSLLKQAGDVEENPGP

SEQ ID NO: 6 - protein, 270 aa, Artificial Sequence
  Synthetic Alpha chain of TCR P1-1
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGVLNFGNEKLTFGTGTRLTIIPIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 7 - protein, 304 aa, Artificial Sequence
  Synthetic Beta chain of TCR P1-1
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSEDTAYYGYTFGSGTRLTVVEEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 8 - protein, 596 aa, Artificial Sequence
  Synthetic TCR P1-1
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSEDTAYYGYTFGSGTRLTVVEEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSGSGATNFSLLKQAGDVEENPGPMLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGVLNFGNEKLTFGTGTRLTIIPIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 9 - DNA, 402 bp, Homo sapiens
atgctgctggaactgatccctctgctgggcatccacttcgtgctgagaaccgccagagcccagagcgtgacccagcccgatatccacatcaccgtgtctgagggcgccagcctggaactgcggtgcaactacagctacggcgccaccccctacctgttttggtacgtgcagagccccggacagggcctgcagctgctgctgaagtactttagcggcgacaccctggtgcagggcatcaagggattcgaggccgagttcaagcggagccagagcagcttcaacctgcggaagccctccgtgcattggagcgacgccgccgagtacttttgtgccgtgggcgtgctgaacttcggcaacgagaagctgacctttggcaccggcacccggctgaccatcatccct

SEQ ID NO: 10 - DNA, 393 bp, Homo sapiens
atgctgtgttctctgctggctctgctgctgggcaccttctttggagtgcggagccagaccatccaccagtggcctgctacactggtgcagcctgtgggcagccctctgagcctggaatgtaccgtggaaggcaccagcaaccccaacctgtactggtacagacaggccgctggcagaggcctgcagctgctgttttacagcgtgggcatcggccagatcagcagcgaggtgccccagaatctgagcgccagcagaccccaggaccggcagtttatcctgagcagcaagaagctgctgctgagcgacagcggcttctacctgtgcgcttggagcgaggacaccgcctactacggctacacctttggcagcggcacccggctgacagtggtggaa

SEQ ID NO: 11 - DNA, 813 bp, Artificial Sequence
  Synthetic Alpha chain of TCR P1-1
atgctgctggaactgatccctctgctgggcatccacttcgtgctgagaaccgccagagcccagagcgtgacccagcccgatatccacatcaccgtgtctgagggcgccagcctggaactgcggtgcaactacagctacggcgccaccccctacctgttttggtacgtgcagagccccggacagggcctgcagctgctgctgaagtactttagcggcgacaccctggtgcagggcatcaagggattcgaggccgagttcaagcggagccagagcagcttcaacctgcggaagccctccgtgcattggagcgacgccgccgagtacttttgtgccgtgggcgtgctgaacttcggcaacgagaagctgacctttggcaccggcacccggctgaccatcatccctatccagaaccccgagcccgccgtgtaccagctgaaggaccccagaagccaggacagcaccctgtgtctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacatgccaggacatcttcaaagagacaaacgccacctacccttccagcgacgtgccctgtgacgccaccctgaccgagaagtccttcgagacagacatgaatctgaatttccagaacctgagcgtgatgggcctgcggatcctgctgctgaaagtggccggcttcaacctgctgatgaccctgcggctgtggtccagctga

SEQ ID NO: 12 - DNA, 912 bp, Artificial Sequence
  Synthetic Beta chain of TCR P1-1
atgctgtgttctctgctggctctgctgctgggcaccttctttggagtgcggagccagaccatccaccagtggcctgctacactggtgcagcctgtgggcagccctctgagcctggaatgtaccgtggaaggcaccagcaaccccaacctgtactggtacagacaggccgctggcagaggcctgcagctgctgttttacagcgtgggcatcggccagatcagcagcgaggtgccccagaatctgagcgccagcagaccccaggaccggcagtttatcctgagcagcaagaagctgctgctgagcgacagcggcttctacctgtgcgcttggagcgaggacaccgcctactacggctacacctttggcagcggcacccggctgacagtggtggaagaagatctgaggaacgtgaccccccccaaggtgtccctgttcgagcccagcaaggccgagatcgccaacaagcagaaagccaccctcgtgtgcctggccagaggcttcttccccgaccacgtggaactgtcctggtgggtcaacggcaaagaggtgcacagcggagtctgcaccgacccccaggcttacaaagagagcaactacagctactgcctgtccagcagactgcgggtgtccgctaccttctggcacaacccccggaaccacttcagatgccaggtgcagttccacggcctgagcgaagaggacaagtggcccgagggcagccccaagcccgtgacccagaatatcagcgccgaggcctggggcagagccgattgtggcatcaccagcgccagctaccaccagggggtgctgagcgccaccatcctgtacgagatcctgctgggcaaggccaccctgtacgccgtgctggtgtccggactggtgctgatggccatggtcaagaagaagaacagc

SEQ ID NO: 13 - DNA, 1791 bp, Artificial Sequence
  Synthetic TCR P1-1
atgctgtgttctctgctggctctgctgctgggcaccttctttggagtgcggagccagaccatccaccagtggcctgctacactggtgcagcctgtgggcagccctctgagcctggaatgtaccgtggaaggcaccagcaaccccaacctgtactggtacagacaggccgctggcagaggcctgcagctgctgttttacagcgtgggcatcggccagatcagcagcgaggtgccccagaatctgagcgccagcagaccccaggaccggcagtttatcctgagcagcaagaagctgctgctgagcgacagcggcttctacctgtgcgcttggagcgaggacaccgcctactacggctacacctttggcagcggcacccggctgacagtggtggaagaagatctgaggaacgtgaccccccccaaggtgtccctgttcgagcccagcaaggccgagatcgccaacaagcagaaagccaccctcgtgtgcctggccagaggcttcttccccgaccacgtggaactgtcctggtgggtcaacggcaaagaggtgcacagcggagtctgcaccgacccccaggcttacaaagagagcaactacagctactgcctgtccagcagactgcgggtgtccgctaccttctggcacaacccccggaaccacttcagatgccaggtgcagttccacggcctgagcgaagaggacaagtggcccgagggcagccccaagcccgtgacccagaatatcagcgccgaggcctggggcagagccgattgtggcatcaccagcgccagctaccaccagggggtgctgagcgccaccatcctgtacgagatcctgctgggcaaggccaccctgtacgccgtgctggtgtccggactggtgctgatggccatggtcaagaagaagaacagcggcagcggcgccaccaacttcagcctgctgaagcaggccggcgacgtggaagagaaccccgggcccatgctgctggaactgatccctctgctgggcatccacttcgtgctgagaaccgccagagcccagagcgtgacccagcccgatatccacatcaccgtgtctgagggcgccagcctggaactgcggtgcaactacagctacggcgccaccccctacctgttttggtacgtgcagagccccggacagggcctgcagctgctgctgaagtactttagcggcgacaccctggtgcagggcatcaagggattcgaggccgagttcaagcggagccagagcagcttcaacctgcggaagccctccgtgcattggagcgacgccgccgagtacttttgtgccgtgggcgtgctgaacttcggcaacgagaagctgacctttggcaccggcacccggctgaccatcatccctatccagaaccccgagcccgccgtgtaccagctgaaggaccccagaagccaggacagcaccctgtgtctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacatgccaggacatcttcaaagagacaaacgccacctacccttccagcgacgtgccctgtgacgccaccctgaccgagaagtccttcgagacagacatgaatctgaatttccagaacctgagcgtgatgggcctgcggatcctgctgctgaaagtggccggcttcaacctgctgatgaccctgcggctgtggtccagctga

SEQ ID NO: 14 - protein, 9 aa, Homo sapiens
FIDKKVLKV

SEQ ID NO: 15 - protein, 580 aa, Homo sapiens
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 16 - DNA, 1743 bp, Homo sapiens
atggccgggaccgtgcgcaccgcgtgcttggtggtggcgatgctgctcagcttggacttcccgggacaggcgcagcccccgccgccgccgccggacgccacctgtcaccaagtccgctccttcttccagagactgcagcccggactcaagtgggtgccagaaactcccgtgccaggatcagatttgcaagtatgtctccctaagggcccaacatgctgctcaagaaagatggaagaaaaataccaactaacagcacgattgaacatggaacagctgcttcagtctgcaagtatggagctcaagttcttaattattcagaatgctgcggttttccaagaggcctttgaaattgttgttcgccatgccaagaactacaccaatgccatgttcaagaacaactacccaagcctgactccacaagcttttgagtttgtgggtgaatttttcacagatgtgtctctctacatcttgggttctgacatcaatgtagatgacatggtcaatgaattgtttgacagcctgtttccagtcatctatacccagctaatgaacccaggcctgcctgattcagccttggacatcaatgagtgcctccgaggagcaagacgtgacctgaaagtatttgggaatttccccaagcttattatgacccaggtttccaagtcactgcaagtcactaggatcttccttcaggctctgaatcttggaattgaagtgatcaacacaactgatcacctgaagttcagtaaggactgtggccgaatgctcaccagaatgtggtactgctcttactgccagggactgatgatggttaaaccctgtggcggttactgcaatgtggtcatgcaaggctgtatggcaggtgtggtggagattgacaagtactggagagaatacattctgtcccttgaagaacttgtgaatggcatgtacagaatctatgacatggagaacgtactgcttggtctcttttcaacaatccatgattctatccagtatgtccagaagaatgcaggaaagctgaccaccactattggcaagttatgtgcccattctcaacaacgccaatatagatctgcttattatcctgaagatctctttattgacaagaaagtattaaaagttgctcatgtagaacatgaagaaaccttatccagccgaagaagggaactaattcagaagttgaagtctttcatcagcttctatagtgctttgcctggctacatctgcagccatagccctgtggcggaaaacgacaccctttgctggaatggacaagaactcgtggagagatacagccaaaaggcagcaaggaatggaatgaaaaaccagttcaatctccatgagctgaaaatgaagggccctgagccagtggtcagtcaaattattgacaaactgaagcacattaaccagctcctgagaaccatgtctatgcccaaaggtagagttctggataaaaacctggatgaggaagggtttgaaagtggagactgcggtgatgatgaagatgagtgcattggaggctctggtgatggaatgataaaagtgaagaatcagctccgcttccttgcagaactggcctatgatctggatgtggatgatgcgcctggaaacagtcagcaggcaactccgaaggacaacgagataagcacctttcacaacctcgggaacgttcattccccgctgaagcttctcaccagcatggccatctcggtggtgtgcttcttcttcctggtgcactga

SEQ ID NO: 17 - protein, 9 aa, Homo sapiens
TIHDSIQYV

SEQ ID NO: 18 - protein, 133 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAKMKTSYDKVIFGPGTSLSVIP

SEQ ID NO: 19 - protein, 142 aa, Homo sapiens
  misc_feature (142)..(142): Xaa can be any naturally occurring amino acid
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAGYSGGGADGLTFGKGTHLIIQPEDLRNVTPX

SEQ ID NO: 20 - protein, 131 aa, Homo sapiens
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASNGQGAQKLVFGQGTRLTIN

SEQ ID NO: 21 - protein, 133 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSPGGIGYEQYFGPGTRLTVTE

SEQ ID NO: 22 - protein, 136 aa, Mus musculus; identical to SEQ ID NO: 3

SEQ ID NO: 23 - protein, 173 aa, Mus musculus; identical to SEQ ID NO: 4

SEQ ID NO: 24 - protein, 269 aa, Artificial Sequence
  Synthetic alpha chain of TCR P2-1 (with variable alpha of SEQ ID NO. 18)
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAKMKTSYDKVIFGPGTSLSVIPIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 25 - protein, 278 aa, Artificial Sequence
  Synthetic alpha chain of TCR P2-1 (with variable alpha of SEQ ID NO. 19)
  misc_feature (142)..(142): Xaa can be any naturally occurring amino acid
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAGYSGGGADGLTFGKGTHLIIQPEDLRNVTPXIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 26 - protein, 267 aa, Artificial Sequence
  Synthetic alpha chain of TCR P2-1 (with variable alpha of SEQ ID NO. 20)
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASNGQGAQKLVFGQGTRLTINIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 27 - protein, 306 aa, Artificial Sequence
  Synthetic beta chain of TCR P2-1
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSPGGIGYEQYFGPGTRLTVTEEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 28 - protein, 597 aa, Artificial Sequence
  Synthetic TCR P2-1 (with variable alpha of SEQ ID NO. 18)
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSPGGIGYEQYFGPGTRLTVTEEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSGSGATNFSLLKQAGDVEENPGPMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAKMKTSYDKVIFGPGTSLSVIPIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 29 - protein, 606 aa, Artificial Sequence
  Synthetic TCR P2-1 (with variable alpha of SEQ ID NO. 19)
  misc_feature (470)..(470): Xaa can be any naturally occurring amino acid
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSPGGIGYEQYFGPGTRLTVTEEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSGSGATNFSLLKQAGDVEENPGPMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAGYSGGGADGLTFGKGTHLIIQPEDLRNVTPXIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 30 - protein, 595 aa, Artificial Sequence
  Synthetic TCR P2-1 (with variable alpha of SEQ ID NO. 20)
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSPGGIGYEQYFGPGTRLTVTEEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSGSGATNFSLLKQAGDVEENPGPMTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASNGQGAQKLVFGQGTRLTINIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 31 - DNA, 399 bp, Homo sapiens
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgcgaaaatgaaaacctcctacgacaaggtgatatttgggccagggacaagcttatcagtcattcca

SEQ ID NO: 32 - DNA, 425 bp, Homo sapiens
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgggtattcaggaggaggtgctgacggactcacctttggcaaagggactcatctaatcatccagcccgaagatctgaggaacgtgaccccccc

SEQ ID NO: 33 - DNA, 393 bp, Homo sapiens
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtaatgggcagggagcccagaagctggtatttggccaaggaaccaggctgactatcaac

SEQ ID NO: 34 - DNA, 399 bp, Homo sapiens
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagccccggggggatcgggtacgagcagtacttcgggccgggcaccaggctcacggtcacagag

SEQ ID NO: 35 - DNA, 810 bp, Artificial Sequence
  Synthetic Alpha chain of TCR P2-1 (with variable alpha of SEQ ID NO. 31)
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgcgaaaatgaaaacctcctacgacaaggtgatatttgggccagggacaagcttatcagtcattccaatccagaaccccgagcccgccgtgtaccagctgaaggaccccagaagccaggacagcaccctgtgtctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacatgccaggacatcttcaaagagacaaacgccacctacccttccagcgacgtgccctgtgacgccaccctgaccgagaagtccttcgagacagacatgaatctgaatttccagaacctgagcgtgatgggcctgcggatcctgctgctgaaagtggccggcttcaacctgctgatgaccctgcggctgtggtccagctga

SEQ ID NO: 36 - DNA, 836 bp, Artificial Sequence
  Synthetic Alpha chain of TCR P2-1 (with variable alpha of SEQ ID NO. 32)
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgggtattcaggaggaggtgctgacggactcacctttggcaaagggactcatctaatcatccagcccgaagatctgaggaacgtgacccccccatccagaaccccgagcccgccgtgtaccagctgaaggaccccagaagccaggacagcaccctgtgtctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacatgccaggacatcttcaaagagacaaacgccacctacccttccagcgacgtgccctgtgacgccaccctgaccgagaagtccttcgagacagacatgaatctgaatttccagaacctgagcgtgatgggcctgcggatcctgctgctgaaagtggccggcttcaacctgctgatgaccctgcggctgtggtccagctga

SEQ ID NO: 37 - DNA, 804 bp, Artificial Sequence
  Synthetic Alpha chain of TCR P2-1 (with variable alpha of SEQ ID NO. 33)
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtaatgggcagggagcccagaagctggtatttggccaaggaaccaggctgactatcaacatccagaaccccgagcccgccgtgtaccagctgaaggaccccagaagccaggacagcaccctgtgtctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacatgccaggacatcttcaaagagacaaacgccacctacccttccagcgacgtgccctgtgacgccaccctgaccgagaagtccttcgagacagacatgaatctgaatttccagaacctgagcgtgatgggcctgcggatcctgctgctgaaagtggccggcttcaacctgctgatgaccctgcggctgtggtccagctga

SEQ ID NO: 38 - DNA, 918 bp, Artificial Sequence
  Synthetic Beta chain of TCR P2-1
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagccccggggggatcgggtacgagcagtacttcgggccgggcaccaggctcacggtcacagaggaagatctgaggaacgtgaccccccccaaggtgtccctgttcgagcccagcaaggccgagatcgccaacaagcagaaagccaccctcgtgtgcctggccagaggcttcttccccgaccacgtggaactgtcctggtgggtcaacggcaaagaggtgcacagcggagtctgcaccgacccccaggcttacaaagagagcaactacagctactgcctgtccagcagactgcgggtgtccgctaccttctggcacaacccccggaaccacttcagatgccaggtgcagttccacggcctgagcgaagaggacaagtggcccgagggcagccccaagcccgtgacccagaatatcagcgccgaggcctggggcagagccgattgtggcatcaccagcgccagctaccaccagggggtgctgagcgccaccatcctgtacgagatcctgctgggcaaggccaccctgtacgccgtgctggtgtccggactggtgctgatggccatggtcaagaagaagaacagc

SEQ ID NO: 39 - DNA, 1794 bp, Artificial Sequence
  Synthetic TCR P2-1 (with variable alpha of SEQ ID NO. 31)
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagccccggggggatcgggtacgagcagtacttcgggccgggcaccaggctcacggtcacagaggaagatctgaggaacgtgaccccccccaaggtgtccctgttcgagcccagcaaggccgagatcgccaacaagcagaaagccaccctcgtgtgcctggccagaggcttcttccccgaccacgtggaactgtcctggtgggtcaacggcaaagaggtgcacagcggagtctgcaccgacccccaggcttacaaagagagcaactacagctactgcctgtccagcagactgcgggtgtccgctaccttctggcacaacccccggaaccacttcagatgccaggtgcagttccacggcctgagcgaagaggacaagtggcccgagggcagccccaagcccgtgacccagaatatcagcgccgaggcctggggcagagccgattgtggcatcaccagcgccagctaccaccagggggtgctgagcgccaccatcctgtacgagatcctgctgggcaaggccaccctgtacgccgtgctggtgtccggactggtgctgatggccatggtcaagaagaagaacagcggcagcggcgccaccaacttcagcctgctgaagcaggccggcgacgtggaagagaaccccgggcccatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgcgaaaatgaaaacctcctacgacaaggtgatatttgggccagggacaagcttatcagtcattccaatccagaaccccgagcccgccgtgtaccagctgaaggaccccagaagccaggacagcaccctgtgtctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacatgccaggacatcttcaaagagacaaacgccacctacccttccagcgacgtgccctgtgacgccaccctgaccgagaagtccttcgagacagacatgaatctgaatttccagaacctgagcgtgatgggcctgcggatcctgctgctgaaagtggccggcttcaacctgctgatgaccctgcggctgtggtccagctga

SEQ ID NO: 40 - DNA, 1820 bp, Artificial Sequence
  Synthetic TCR P2-1 (with variable alpha of SEQ ID NO. 32)
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagccccggggggatcgggtacgagcagtacttcgggccgggcaccaggctcacggtcacagaggaagatctgaggaacgtgaccccccccaaggtgtccctgttcgagcccagcaaggccgagatcgccaacaagcagaaagccaccctcgtgtgcctggccagaggcttcttccccgaccacgtggaactgtcctggtgggtcaacggcaaagaggtgcacagcggagtctgcaccgacccccaggcttacaaagagagcaactacagctactgcctgtccagcagactgcgggtgtccgctaccttctggcacaacccccggaaccacttcagatgccaggtgcagttccacggcctgagcgaagaggacaagtggcccgagggcagccccaagcccgtgacccagaatatcagcgccgaggcctggggcagagccgattgtggcatcaccagcgccagctaccaccagggggtgctgagcgccaccatcctgtacgagatcctgctgggcaaggccaccctgtacgccgtgctggtgtccggactggtgctgatggccatggtcaagaagaagaacagcggcagcggcgccaccaacttcagcctgctgaagcaggccggcgacgtggaagagaaccccgggcccatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgggtattcaggaggaggtgctgacggactcacctttggcaaagggactcatctaatcatccagcccgaagatctgaggaacgtgacccccccatccagaaccccgagcccgccgtgtaccagctgaaggaccccagaagccaggacagcaccctgtgtctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacatgccaggacatcttcaaagagacaaacgccacctacccttccagcgacgtgccctgtgacgccaccctgaccgagaagtccttcgagacagacatgaatctgaatttccagaacctgagcgtgatgggcctgcggatcctgctgctgaaagtggccggcttcaacctgctgatgaccctgcggctgtggtccagctga

SEQ ID NO: 41 - DNA, 1788 bp, Artificial Sequence
  Synthetic TCR P2-1 (with variable alpha of SEQ ID NO. 33)
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagccccggggggatcgggtacgagcagtacttcgggccgggcaccaggctcacggtcacagaggaagatctgaggaacgtgaccccccccaaggtgtccctgttcgagcccagcaaggccgagatcgccaacaagcagaaagccaccctcgtgtgcctggccagaggcttcttccccgaccacgtggaactgtcctggtgggtcaacggcaaagaggtgcacagcggagtctgcaccgacccccaggcttacaaagagagcaactacagctactgcctgtccagcagactgcgggtgtccgctaccttctggcacaacccccggaaccacttcagatgccaggtgcagttccacggcctgagcgaagaggacaagtggcccgagggcagccccaagcccgtgacccagaatatcagcgccgaggcctggggcagagccgattgtggcatcaccagcgccagctaccaccagggggtgctgagcgccaccatcctgtacgagatcctgctgggcaaggccaccctgtacgccgtgctggtgtccggactggtgctgatggccatggtcaagaagaagaacagcggcagcggcgccaccaacttcagcctgctgaagcaggccggcgacgtggaagagaaccccgggcccatgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtaatgggcagggagcccagaagctggtatttggccaaggaaccaggctgactatcaacatccagaaccccgagcccgccgtgtaccagctgaaggaccccagaagccaggacagcaccctgtgtctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacatgccaggacatcttcaaagagacaaacgccacctacccttccagcgacgtgccctgtgacgccaccctgaccgagaagtccttcgagacagacatgaatctgaatttccagaacctgagcgtgatgggcctgcggatcctgctgctgaaagtggccggcttcaacctgctgatgaccctgcggctgtggtccagctga

SEQ ID NO: 42 - DNA, 18 bp, Artificial Sequence
  Synthetic Primer (with R = A or G; D = A, G or T)
tgagtgtcccrgarggdr

SEQ ID NO: 43 - DNA, 22 bp, Artificial Sequence
  Synthetic Primer
gtgacacatttgtttgagaatc

SEQ ID NO: 44 - DNA, 20 bp, Artificial Sequence
  Synthetic Primer
  misc_feature (3)..(4): N = inosine
  misc_feature (8)..(8): N = inosine
gcnntktnytggtaymgaca

SEQ ID NO: 45 - DNA, 20 bp, Artificial Sequence
  Synthetic Primer
  misc_feature (3)..(3): N = inosine
  misc_feature (16)..(16): N = inosine
ctntktwttggtaycnkcag

SEQ ID NO: 46 - DNA, 19 bp, Artificial Sequence
  Synthetic Primer
gcacctccttcccattcac

(19) United States
(12) Patent Application Publication
(10) Pub. No.: US 2017/0267737 A1
(43) Pub. Date: Sep. 21, 2017

(54) Glypican-3-specific T-cell receptors and their uses for immunotherapy of hepatocellular carcinoma

(71) Applicant: Technische Universität München, München (DE)

(72) Inventors: Ulrike Protzer, Munich (DE); Christina Dargel, Munich (DE)

(73) Assignee: Technische Universität München, München (DE)

(21) Appl. No.: 15/310,727

(22) PCT Filed: May 7, 2015
(86) PCT No.: PCT/EP2015/060065

§ 371 Date: Nov. 11, 2016

(30) Foreign Application Priority Data
May 13, 2014 (EP) 14168107.2

(51) Int. Cl. C07K 14/705; C07K 16/18; A61K 39/00; A61K 35/17; G01N 33/574; C07K 14/725; C12N 5/0783

(52) U.S. Cl. CPC C07K 14/705; C07K 14/7051; C07K 16/18; C12N 5/0636; A61K 35/17; G01N 33/57438; A61K 39/0011; C07K 2317/31; C07K 2317/622; C12N 2510/00; G01N 2333/705; C07K 2319/70

(57) ABSTRACT

The present invention relates to glypican-3-specific T-cell receptors. The present invention further relates to soluble TCR constructs, chimeric TCRs, bi-specific antibodies, nucleic acids, expression constructs and cells comprising said TCRs or TCR constructs. The present invention further relates to the use of the TCR or the soluble TCR constructs or chimeric TCRs or bi-specific antibodies as a medicament, preferably in the detection, diagnosis, prognosis, prevention and/or treatment of liver cancer, in particular hepatocellular carcinoma, or other cancers expressing GPC3. The present invention further relates to methods of detecting, diagnosing, prognosing, preventing and/or treating liver cancer, in particular hepatocellular carcinoma, or other cancers expressing GPC3. The present invention further relates to peptides comprising glypican-3 epitope(s) and respective nucleic acids encoding them, antibodies and compositions as well as their use as (peptide) vaccines. The present invention further relates to vaccines comprising the peptide(s).

The present invention relates to glypican-3-specific T-cell receptors. The present invention further relates to soluble TCR constructs, chimeric TCRs, bi-specific antibodies, nucleic acids, expression constructs and cells comprising said TCRs or TCR constructs. The present invention further relates to the use of the TCR or the soluble TCR constructs or chimeric TCRs or bi-specific antibodies as a medicament, preferably in the detection, diagnosis, prognosis, prevention and/or treatment of liver cancer, in particular hepatocellular carcinoma, or other cancers expressing GPC3. The present invention further relates to methods of detecting, diagnosing, prognosing, preventing and/or treating liver cancer, in particular hepatocellular carcinoma, or other cancers expressing GPC3. The present invention further relates to peptides comprising glypican-3 epitope(s) and respective nucleic acids encoding them, antibodies and compositions as well as their use as (peptide) vaccines. The present invention further relates to vaccines comprising the peptide(s).

### BACKGROUND OF THE INVENTION

Hepatocellular carcinoma (HCC) is the most common primary liver cancer and the third most common cause of cancer related death worldwide. Patients chronically infected with hepatitis B or C are at high risk for development of HCC, but alcohol abuses and metabolic disorders can also lead to HCC development.

Less than 40% of patients fulfil the criteria for curative treatment like tumour resection or liver transplantation. Furthermore, the risk of tumour recurrence is high. Patients with advanced HCC can only undergo palliative treatment such as local ablation or the multikinase inhibitor sorafenib. Nevertheless these treatments result in a marginal beneficial effect on the expected lifetime of patients. As a result, the prognosis of HCC remains poor and new therapies are urgently needed.

WO 2012/070468 A1 discloses a glypican-3 (GPC3) specific antibody and a human chimeric antigen receptor (CAR) able to target GPC3. WO 2012/070468 A1 further discloses compositions and methods for diagnosing and treating diseases associated with dysregulated expression of GPC3, such as liver cancer, using said antibodies.

There is a need in the art for improved means and methods for detecting, diagnosing, prognosing, preventing and/or treating liver cancer, in particular hepatocellular carcinoma (HCC), such as via specific T cells or by immunotherapy or peptide-based vaccines.

### SUMMARY OF THE INVENTION

According to the present invention this object is solved by a T-cell receptor (TCR) comprising:

(i) a T cell receptor α-chain comprising the amino acid sequence of SEQ ID NO. 1 (variable alpha)
or an amino acid sequence that has at least 80% identity to the amino acid sequence of SEQ ID NO. 1, more preferably at least 85% identity, more preferably 90% or 95% identity, or any variation of the TCR sequence provided that said variation retains its functional ability to bind to the epitope with the amino acid sequence of SEQ ID NO. 14 (GPC3 peptide 367) or to its HLA-A2 bound form
and/or
(ii) a T-cell receptor β-chain comprising the amino acid sequence of SEQ ID NO. 2 (variable beta)
or an amino acid sequence that has at least 80% identity to the amino acid sequence of SEQ ID NO. 2, more preferably at least 85% identity, more preferably 90% or 95% identity,
or any variation of the TCR sequence provided that said variation retains its functional ability to bind to the epitope with the amino acid sequence of SEQ ID NO. 14 (GPC3 peptide 367) or to its HLA-A2 bound form.

According to the present invention this object is solved by a T-cell receptor (TCR) comprising:

(i) a T cell receptor α-chain comprising an amino acid sequence selected from the group of SEQ ID NOs. 18-20 (3× variable alpha)
or an amino acid sequence that has at least 80% identity to the amino acid sequence of SEQ ID NO. 1, more preferably at least 85% identity, more preferably 90% or 95% identity, or any variation of the TCR sequence provided that said variation retains its functional ability to bind to the epitope with the amino acid sequence of SEQ ID NO. 17 (GPC3 peptide 326) or to its HLA-A2 bound form
and/or
(ii) a T-cell receptor β-chain comprising the amino acid sequence of SEQ ID NO. 21 (variable beta)
or an amino acid sequence that has at least 80% identity to the amino acid sequence of SEQ ID NO. 2, more preferably at least 85% identity, more preferably 90% or 95% identity, or any variation of the TCR sequence provided that said variation retains its functional ability to bind to the epitope with the amino acid sequence of SEQ ID NO. 17 (GPC3 peptide 326) or to its HLA-A2 bound form.

According to the present invention this object is solved by a soluble T-cell receptor (sTCR) construct comprising

(1) at least one of T-cell receptor α-chain(s) as defined herein, and/or T-cell receptor β-chain(s) as defined herein,
preferably covalently linked to each other to form TCR heterodimers or multimers, and (2) fusion component(s)
preferably selected from Fc receptors and/or Fc domains, cytokines, such as IL-2 or IL-15, toxins, antibodies, such as anti-CD3, anti-CD28, anti-CD5, anti-CD16 or anti-CD56 antibodies, or combinations thereof,
   wherein the at least one T-cell receptor chain (1) is bound to the fusion component(s) (2),

According to the present invention this object is solved by a chimeric T-cell receptor comprising

at least one of
the T-cell receptor α-chain as defined herein,
the T-cell receptor β-chain as defined herein, or
the T-cell receptor according to the present invention,
   wherein the TCR α-chain and/or the TCR β-chain is/are fused to CD3-zeta chain(s) and/or other TCR stimulation domains, such as the intracellular CD28, CD137 or CD134 domain, preferably via a linker.

According to the present invention this object is solved by a bi-specific antibody comprising

(a) the T-cell receptor α- and β-chain(s) of the TCR defined herein,
which are linked with each other
and fused, preferably via a linker, to
   (b) an antibody or a single chain antibody fragment (scFv) which is directed against an antigen or epitope on the surface of lymphocytes.

According to the present invention this object is solved by a nucleic acid encoding the T-cell receptor according to the present invention or encoding the soluble T-cell receptor construct according to the present invention or encoding the chimeric T-cell receptor according to the present invention or encoding the bi-specific antibody according to the present invention.

According to the present invention this object is solved by an expression construct for expressing the T-cell receptor or soluble T-cell receptor construct or the chimeric T-cell receptor or the bi-specific antibody according to the present invention in a cell.

According to the present invention this object is solved by a cell comprising the T-cell receptor or soluble T-cell receptor construct or chimeric T-cell receptor or bi-specific antibody according to the present invention or the nucleic acid(s) according to the present invention or the expression construct according to the present invention.

According to the present invention this object is solved by a pharmaceutical composition comprising one or more of:
(i) the T-cell receptor of the present invention;
(ii) the soluble T-cell receptor construct of the present invention;
(iii) the chimeric T-cell receptor of the present invention;
(iv) the bi-specific antibody of the present invention;
(v) the nucleic acid(s) of the present invention or the expression construct of the present invention; and/or
(vi) the cell of the present invention,
and, optionally, pharmaceutically excipient(s).

According to the present invention this object is solved by the use of a T-cell receptor of the present invention, a chimeric T-cell receptor of the present invention, a bi-specific antibody of the present invention, a nucleic acid of the present invention or an expression construct of the present invention for generating genetically modified lymphocytes.

According to the present invention this object is solved by providing the T-cell receptor of the present invention, the chimeric T-cell receptor of the present invention, the bi-specific antibody of the present invention, the nucleic acid of the present invention or the expression construct of the present invention for use as a medicament.

According to the present invention this object is solved by providing the T-cell receptor of the present invention, the chimeric T-cell receptor of the present invention, the bi-specific antibody of the present invention, the nucleic acid of the present invention or the expression construct of the present invention for use in the detection, diagnosis, prognosis, prevention and/or treatment of liver cancer, in particular hepatocellular carcinoma (HCC), or other cancers expressing GPC3.

According to the present invention this object is solved a method of preventing and/or treating liver cancer, in particular hepatocellular carcinoma (HCC), or other cancers expressing GPC3, comprising the steps of

(a) providing lymphocytes of a patient or a blood donor;
(b) providing one or more of
(i) a T-cell receptor of the present invention or a soluble T-cell receptor construct of the present invention or a chimeric T-cell receptor of the present invention or a bi-specific antibody of the present invention,
(ii) a nucleic acid of the present invention,
(iii) an expression construct of the present invention,
(iv) a cell of the present invention, and
(v) a pharmaceutical composition of the present invention;
   (c) ex vivo introduction of one or more of (i) to (v) of step (b) into the lymphocytes of step (a) and, thereby, obtaining genetically modified lymphocytes,
   (d) administering the genetically modified lymphocytes of step (c) to a subject or patient in need thereof;
   (e) optional, combining any of the steps (b) to (d) with radiation, checkpoint inhibitor or cancer chemotherapy.

According to the present invention this object is solved a method of preventing and/or treating liver cancer, in particular hepatocellular carcinoma (HCC), or other cancers expressing GPC3, comprising the steps of

(a) providing one or more of
(i) a T-cell receptor of the present invention or a soluble T-cell receptor construct of the present invention or a chimeric T-cell receptor of the present invention or a bi-specific antibody of the present invention,
(ii) a nucleic acid of the present invention,
(iii) an expression construct of the present invention,
(iv) a cell of the present invention, and
(v) a pharmaceutical composition of the present invention;
   (b) direct application, preferably via injection or infusion, of one or more of (i) to (v) of step (a) to a subject or patient in need thereof,
   (c) optional, combining step (b) with radiation, checkpoint inhibitor or cancer chemotherapy.

According to the present invention this object is solved a method of detecting, diagnosing, prognosing, preventing and/or treating liver cancer, in particular hepatocellular carcinoma (HCC), or other cancers expressing GPC3,

comprising the detection and/or destruction of (liver) cancer cells, in particular hepatocellular carcinoma (HCC) cells, of a patient with the use of the soluble T-cell receptor construct of the present invention.

According to the present invention this object is solved by a peptide comprising at least one GPC3 epitope comprising the amino acid sequence of SEQ ID NO. 13 (GPC3 peptide 367).

According to the present invention this object is solved by a nucleic acid molecule coding for at least one peptide according to the present invention or a plasmid comprising at least one such nucleic acid molecule.

According to the present invention this object is solved by an antibody against a peptide according to the present invention.

According to the present invention this object is solved by a composition comprising

(i) at least one peptide according to the present invention or at least one nucleic acid according to the present invention,
(ii) optionally, a carrier,
(iii) optionally, an adjuvant.

According to the present invention this object is solved by providing the peptide of the present invention, the nucleic acid molecule of the present invention or the pharmaceutical composition of the present invention for use in medicine.

According to the present invention this object is solved by providing the peptide of the present invention, the nucleic acid molecule of the present invention or the pharmaceutical composition of the present invention for use as a vaccine.

According to the present invention this object is solved by a vaccine comprising

(i) at least one peptide according to the present invention or at least one nucleic acid according to the present invention or a composition of the present invention,
(ii) optionally, an excipient.

According to the present invention this object is solved by the use of the vaccine of the present invention for preventing and/or treating liver cancer, in particular hepatocellular carcinoma (HCC), or other cancers expressing GPC3.

According to the present invention this object is solved by a method of preventing and/or treating liver cancer, in particular hepatocellular carcinoma (HCC), or other cancers expressing GPC3,

comprising the step of administering a peptide of the present invention, a nucleic acid molecule of the present invention, a pharmaceutical composition of the present invention or a vaccine of the present invention to a subject in need thereof.

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “5 to 50 amino acids” should be interpreted to include not only the explicitly recited values of 5 to 50, but also include individual values and sub-ranges within the indicated range. Thus, included in this numerical range are individual values such as 5, 6, 7, 8, 9, 10, 11, 12, 13, 14, 15, 16, 17, 18, 19, 20, . . . 48, 49, 50 and sub-ranges such as from 9 to 50, from 5 to 25, from 9 to 25, from 10 to 25, from 10 to 20 and from 15 to 25, etc. This same principle applies to ranges reciting only one numerical value, such as “at least 8 amino acids”. Furthermore, such an interpretation should apply regardless of the breadth of the range or the characteristics being described.

### GPC3-Specific T-Cell Receptors

As discussed above, the present invention provides GPC3-specific T-cell receptors (TCRs).

A majority of T cells have a T cell receptor (TCR) existing as a complex of several proteins. The actual T cell receptor is composed of two (separate) peptide chains, which are produced from the independent T cell receptor alpha and beta (TCR α and TCR β) genes and are called α- and β-TCR chains. γδ T cells (gamma delta T cells) represent a small subset of T cells that possess a distinct T cell receptor (TCR) on their surface. However, in γδ T cells, the TCR is made up of one γ-chain and one δ-chain. This group of T cells is much less common (2% of total T cells) than the αβ T cells.

The structure of the T cell receptor is very similar to immunoglobulin Fab fragments, which are regions defined as the combined light and heavy chain of an antibody arm. Each chain of the TCR is a member of the immunoglobulin superfamily and possesses one N-terminal inununoglobulin (Ig)-variable (V) domain, one Ig-constant (C) domain, a transmembrane/cell membrane-spanning region, and a short cytoplasmic tail at the C-terminal end.

According to the invention, the term “variable region of a T cell receptor” relates to the variable domains of the TCR chains. The variable domain of both the TCR α-chain and β-chain have three hypervariable or complementarity determining regions (CDRs), whereas the variable region of the β-chain has an additional area of hypervariability (HV4) that does not normally contact antigen and therefore is not considered a CDR.

The first signal in activation of T cells is provided by binding of the T cell receptor to a short peptide presented by the major histocompatibility complex (MHC) on another cell. This ensures that only a T cell with a TCR specific to that peptide is activated.

HCC expresses like many other tumours tumour-associated antigens. Glypican-3 (GPC3) and alpha-fetoprotein are not expressed in healthy human liver but reactivated in up to 70% and 80% of all HCCs, respectively.

### —TCR Specific for GPC3 Peptide 367

The inventors have now identified immunodominant GPC3 epitopes, namely the HLA-A2 bound GPC3 peptide 367, and cloned a TCR that is specific for GPC3 peptide 367.

Preferably, the GPC3-specific T-cell receptor (TCR) of the present invention

(1) comprises an alpha and a beta chain each comprising variable and constant domain(s),
(2) comprises codon-optimized variable domains for higher expression levels,
(3) is murinized, i.e. comprises murine constant domains, which helps to avoid miss pairing with endogenous TCR chains,
and/or
(4) comprises a linker or hinge region to link the alpha and beta chain.

As discussed above, the GPC3-specific TCR of the present invention comprises:

(i) a T cell receptor α-chain comprising the amino acid sequence of SEQ ID NO. 1 (variable alpha)
or an amino acid sequence that has at least 80% identity to the amino acid sequence of SEQ ID NO. 1, more preferably at least 85% identity, more preferably 90% or 95% identity,
or any variation of the TCR sequence provided that said variation retains its functional ability to bind to the epitope with the amino acid sequence of SEQ ID NO. 14 (GPC3 peptide 367) or to its HLA-A2 bound form
and/or
   (ii) a T-cell receptor β-chain comprising the amino acid sequence of SEQ ID NO. 2 (variable beta)
or an amino acid sequence that has at least 80% identity to the amino acid sequence of SEQ in NO. 2, more preferably at least 85% identity, more preferably 90% or 95% identity,
or any variation of the TCR sequence provided that said variation retains its functional ability to bind to the epitope with the amino acid sequence of SEQ ID NO. 14 (GPC3 peptide 367) or to its HLA-A2 bound form.

The terms “any variation of the TCR sequence provided that the biological activity is retained” or “any variation of the TCR sequence provided that said variation retains its functional ability to bind to the epitope with the amino acid sequence of SEQ ID NO. 14 (GPC3 peptide 367) or to its HLA-A2 bound form” as interchangeably used herein refers to amino acid sequences of the TCR chain(s)/receptor(s) of the present invention that retain their functional activity with regard to TCR recognition of the GPC3 peptide 367, preferably bound to the HLA A2-molecule.

In a preferred embodiment, the T-cell receptor (TCR) of the present invention comprises

(i) a T cell receptor α-chain further comprising the amino acid sequence of SEQ ID NO. 3 (murine alpha)
or an amino acid sequence that has at least 80% identity to the amino acid sequence of SEQ ID NO. 3, more preferably at least 85% identity, more preferably 90% or 95% identity, and/or
   (ii) a T-cell receptor β-chain further comprising the amino acid sequence of SEQ ID NO. 4 (murine beta)
or an amino acid sequence that has at least 80% identity to the amino acid sequence of SEQ ID NO. 4, more preferably at least 85% identity, more preferably 90% or 95% identity.

In a preferred embodiment, the T-cell receptor (TCR) of the present invention comprises

(iii) a linker to hinge region comprising the amino acid sequence of SEQ ID NO. 5 (P2A) or an amino acid sequence that has at least 80% identity to the amino acid sequence of SEQ ID NO. 5.

In a preferred embodiment, the T-cell receptor (TCR) of the present invention comprises

(i) a T cell receptor α-chain comprising the amino acid sequence of SEQ ID NO. 1 (variable alpha) and SEQ ID NO. 3 (murine alpha),
preferably comprising or consisting of the amino acid sequence of SEQ ID NO. 6 (variable alpha+murine alpha)
or an amino acid sequence that has at least 80% identity to the amino acid sequence of SEQ ID NO. 6, more preferably at least 85% identity, more preferably 90% or 95% identity,
   or any variation of the TCR sequence provided that said variation retains its functional ability to bind to the epitope with the amino acid sequence of SEQ ID NO. 14 (GPC3 peptide 367) or to its HLA-A2 bound form
   and/or
   (ii) a T-cell receptor β-chain comprising the amino acid sequence of SEQ ID NO. 2 (variable beta) and SEQ ID NO. 4 (murine beta),
   preferably comprising or consisting of the amino acid sequence of SEQ ID NO. 7 (variable beta+murine beta)
or an amino acid sequence that has at least 80% identity to the amino acid sequence of SEQ ID NO. 7, more preferably at least 85% identity, more preferably 90% or 95% identity,
   or any variation of the TCR sequence provided that said variation retains its functional ability to bind to the epitope with the amino acid sequence of SEQ ID NO. 14 (GPC3 peptide 367) or to its HLA-A2 bound form.

In a preferred embodiment, the T-cell receptor (TCR) of the present invention comprises or consists of the amino acid sequence of SEQ ID NO. 8 (aa sequence of the entire TCR P1-1).

SEQ ID NO. 1 shows the amino acid sequence of the variable region of the T-cell receptor alpha-chain (variable alpha)

MLLELIPLLGIHFVLRTARAQSVTQPDIHITVSEGASLELRCNYSYGATPY
LFWYVQSPGQGLQLLLKYFSGDTLVQGIKGFEAEFKRSQSSFNLRKPSVHW
SDAAEYFCAVGVLNFGNEKLTFGTGTRLTIIP

SEQ ID NO. 2 shows the amino acid sequence of the variable region of the T-cell receptor beta-chain (variable beta)

MLCSLLALLLGTFFGVRSQTIHQWPATLVQPVGSPLSLECTVEGTSNPNLY
WYRQAAGRGLQLLFYSVGIGQISSEVPQNLSASRPQDRQFILSSKKLLLSD
SGFYLCAWSEDTAYYGYTFGSGTRLTVVE

SEQ ID NO. 3 shows the amino acid sequence of the constant region of the T-cell receptor alpha-chain, which comprises murine constant domains (murine alpha)

IQNPEPAVYQLKDPRSQDSTLCLFTDFDSQINVPKTMESGTFITDKCVLD
MKAMDSKSNGAIAWSNQTSFTCQDIFKETNATYPSSDVPCDATLTEKSFE
TDMNLNFQNLSVMGLRILLLKVAGFNLLMTLRLWSS

SEQ ID NO. 4 shows the amino acid sequence of the constant region of the T-cell receptor beta-chain, which comprises murine constant domains (murine beta)

EDLRNVTPPKVSLFEPSKAEIANKQKATLVCLARGFFPDHVELSWWVNGK
EVHSGVCTDPQAYKESNYSYCLSSRLRVSATFWHNPRNHFRCQVQFHGLS
EEDKWPEGSPKPVTQNISAEAWGRADCGITSASYHQGVLSATILYEILLG
KATLYAVLVSGLVLMAMVKKKNS

SEQ ID NO. 5 shows the amino acid sequence of the linker or hinge region which links the beta chain and alpha chain:

GSGATNFSLLKQAGDVEENPGP

SEQ ID NO. 6 shows the full length amino acid sequence of the T-cell receptor alpha-chain (wherein SEQ ID NO. 1 is underlined):

MLLELIPLLGIHFVLRTARAQSVTQPDIHITVSEGASLELRCNYSYGATP
YLFWYVQSPGQGLQLLLKYFSGDTLVQGIKGFEAEFKRSQSSFNLRKPSV
HWSDAAEYFCAVGVLNFGNEKLTFGTGTRLTIIPIQNPEPAVYQLKDPRS
QDSTLCLFTDFDSQINVPKTMESGTFITDKCVLDMKAMDSKSNGAIAWSN
QTSFTCQDIFKETNATYPSSDVPCDATLTEKSFETDMNLNFQNLSVMGLR
ILLLKVAGFNLLMTLRLWSS

SEQ ID NO. 7 shows the full length amino acid sequence of the T-cell receptor beta-chain (wherein SEQ ID NO. 2 is underlined):

MLCSLLALLLGTFFGVRSQTIHQWPATLVQPVGSPLSLECTVEGTSNPNL
YWYRQAAGRGLQLLFYSVGIGQISSEVPQNLSASRPQDRQFILSSKKLLL
SDSGFYLCAWSEDTAYYGYTFGSGTRLTVVEEDLRNVTPPKVSLFEPSKA
EIANKQKATLVCLARGFFPDHVELSWWVNGKEVHSGVCTDPQAYKESNYS
YCLSSRLRVSATFWHNPRNHFRCQVQFHGLSEEDKWPEGSPKPVTQNISA
EAWGRADCGITSASYHQGVLSATILYEILLGKATLYAVLVSGLVLMAMVK
KKNS

SEQ ID NO. 8 shows the full length amino acid sequence of the T-cell receptor, including beta chain, linker and alpha chain:

MLCSLLALLLGTFFGVRSQTIHQWPATLVQPVGSPLSLECTVEGTSNPNL
YWYRQAAGRGLQLLFYSVGIGQISSEVPQNLSASRPQDRQFILSSKKLLL
SDSGFYLCAWSEDTAYYGYTFGSGTRLTVVEEDLRNVTPPKVSLFEPSKA
EIANKQKATLVCLARGFFPDHVELSWWVNGKEVHSGVCTDPQAYKESNYS
YCLSSRLRVSATFWHNPRNHFRCQVQFHGLSEEDKWPEGSPKPVTQNISA
EAWGRADCGITSASYHQGVLSATILYEILLGKATLYAVLVSGLVLMAMVK
KKNSGSGATNFSLLKQAGDVEENPGPMLLELIPLLGIHFVLRTARAQSVT
QPDIHITVSEGASLELRCNYSYGATPYLFWYVQSPGQGLQLLLKYFSGDT
LVQGIKGFEAEFKRSQSSFNLRKPSVHWSDAAEYFCAVGVLNFGNEKLTF
GTGTRLTIIPIQNPEPAVYQLKDPRSQDSTLCLFTDFDSQINVPKTMESG
TFITDKCVLDMKAMDSKSNGAIAWSNQTSFTCQDIFKETNATYPSSDVPC
DATLTEKSFETDMNLNFQNLSVMGLRILLLKVAGFNLLMTLRLWSS*

### —TCR Specific for GPC3 Peptide 326

The inventors have furthermore cloned a TCR that is specific for GPC3 peptide 326.

Preferably, the GPC3-specific T-cell receptor (TCR) of the present invention

(1) comprises an alpha and a beta chain each comprising variable and constant domain(s),
(2) comprises codon-optimized variable domains for higher expression levels,
(3) is murinized, i.e. comprises murine constant domains, which helps to avoid miss pairing with endogenous TCR chains,
and/or
(4) comprises a linker or hinge region to link the alpha and beta chain.

As discussed above, the GPC3-specific TCR of the present invention comprises:

(i) a T cell receptor α-chain comprising an amino acid sequence selected from the group of SEQ ID NOs. 18-20 (3× variable alpha)
or an amino acid sequence that has at least 80% identity to the amino acid sequence of SEQ ID NO. 1, more preferably at least 85% identity, more preferably 90% or 95% identity,
or any variation of the TCR sequence provided that said variation retains its functional ability to bind to the epitope with the amino acid sequence of SEQ ID NO. 17 (GPC3 peptide 326) or to its HLA-A2 bound form
and/or
   (ii) a T-cell receptor β-chain comprising the amino acid sequence of SEQ ID NO. 21 (variable beta)
or an amino acid sequence that has at least 80% identity to the amino acid sequence of SEQ ID NO. 2, more preferably at least 85% identity, more preferably 90% or 95% identity,
or any variation of the TCR sequence provided that said variation retains its functional ability to bind to the epitope with the amino acid sequence of SEQ ID NO. 17 (GPC3 peptide 326) or to its HLA-A2 bound form.

The terms “any variation of the TCR sequence provided that the biological activity is retained” or “any variation of the TCR sequence provided that said variation retains its functional ability to bind to the epitope with the amino acid sequence of SEQ ID NO. 17 (GPC3 peptide 326) or to its HLA-A2 bound form” as interchangeably used herein refers to amino acid sequences of the TCR chain(s)/receptor(s) of the present invention that retain their functional activity with regard to TCR recognition of the GPC3 peptide 326, preferably bound to the HLA A2-molecule.

In a preferred embodiment, the T-cell receptor (TCR) of the present invention comprises

(i) a T cell receptor α-chain further comprising an amino acid sequence selected from the group of SEQ ID NOs. 22-24 (3× murine alpha)
or an amino acid sequence that has at least 80% identity to the amino acid sequence of SEQ ID NO. 3, more preferably at least 85% identity, more preferably 90% or 95% identity,
and/or
   (ii) a T-cell receptor β-chain further comprising the amino acid sequence of SEQ ID NO. 25 (murine beta)
or an amino acid sequence that has at least 80% identity to the amino acid sequence of SEQ ID NO. 4, more preferably at least 85% identity, more preferably 90% or 95% identity.

In a preferred embodiment, the T-cell receptor (TCR) of the present invention comprises

(iii) a linker to hinge region comprising the amino acid sequence of SEQ ID NO. 5 (P2A) or an amino acid sequence that has at least 80% identity to the amino acid sequence of SEQ ID NO. 5.

In a preferred embodiment, the T-cell receptor (TCR) of the present invention comprises

(i) a T cell receptor α-chain comprising an amino acid sequence selected from the group of
(i-1) the amino acid sequence of SEQ ID NO. 18 (variable alpha) and SEQ ID NO. 22 (murine alpha),
preferably comprising or consisting of the amino acid sequence of SEQ ID NO. 26 (variable alpha+murine alpha)
or an amino acid sequence that has at least 80% identity to the amino acid sequence of SEQ ID NO. 26, more preferably at least 85% identity, more preferably 90% or 95% identity,
(i-2) the amino acid sequence of SEQ ID NO. 19 (variable alpha) and SEQ ID NO. 23 (murine alpha),
preferably comprising or consisting of the amino acid sequence of SEQ ID NO. 27 (variable alpha+murine alpha)
or an amino acid sequence that has at least 80% identity to the amino acid sequence of SEQ ID NO. 27, more preferably at least 85% identity, more preferably 90% or 95% identity,
(i-3) the amino acid sequence of SEQ ID NO. 20 (variable alpha) and SEQ ID NO. 24 (murine alpha),
preferably comprising or consisting of the amino acid sequence of SEQ ID NO. 28 (variable alpha+murine alpha)
or an amino acid sequence that has at least 80% identity to the amino acid sequence of SEQ ID NO. 28, more preferably at least 85% identity, more preferably 90% or 95% identity,
   or any variation of the TCR sequence provided that said variation retains its functional ability to bind to the epitope with the amino acid sequence of SEQ ID NO. 17 (GPC3 peptide 326) or to its HLA-A2 bound form
   and/or
   (ii) a T-cell receptor β-chain comprising the amino acid sequence of SEQ ID NO. 21 (variable beta) and SEQ ID NO. 25 (murine beta),
   preferably comprising or consisting of the amino acid sequence of SEQ ID NO. 29 (variable beta+murine beta)
or an amino acid sequence that has at least 80% identity to the amino acid sequence of SEQ ID NO. 29, more preferably at least 85% identity, more preferably 90% or 95% identity,
   or any variation of the TCR sequence provided that said variation retains its functional ability to bind to the epitope with the amino acid sequence of SEQ ID NO. 17 (GPC3 peptide 326) or to its HLA-A2 bound form.

In a preferred embodiment, the T-cell receptor (TCR) of the present invention comprises or consists of an amino acid sequence selected from the group of SEQ ID NOs. 30-32 (3× aa sequence of the entire TCR P2-1).

SEQ ID NO. 18 shows the amino acid sequence of the variable region of one T-cell receptor alpha-chain (variable alpha)

MKSLRVLLVILWLQLSWVWSQQKEVEQNSGPLSVPEGAIASLNCTYSDRG
SQSFEWYRQYSGKSPELIMFIYSNGDKEDGRFTAQLNKASQYVSLLIRDS
QPSDSATYLCAAKMKTSYDKVIFGPGTSLSVIP

SEQ ID NO. 19 shows the amino acid sequence of the variable region of another T-cell receptor alpha-chain (variable alpha)

MKSLRVLLVILWLQLSWVWSQQKEVEQNSGPLSVPEGAIASLNCTYSDRG
SQSFFWYRQYSGKSPELIMFIYSNGDKEDGRFTAQLNKASQYVSLLIRDS
QPSDSATYLCAGYSGGGADGLTFGKGTHLIIQPEDLRNVTPX

SEQ ID NO. 20 shows the amino acid sequence of the variable region of another T-cell receptor alpha-chain (variable alpha)

MTSIRAVFIFLWLQLDLVNGENVEQHPSTLSVQEGDSAVIKCTYSDSASN
YFPWYKQELGKRPQLIIDIRSNVGEKKDQRIAVTLNKTAKHFSLHITETQ
PEDSAVYFCAASNGQGAQKLVFGQGTRLTIN

SEQ ID NO. 21 shows the amino acid sequence of the variable region of the T-cell receptor beta-chain (variable beta)

MGFRLLCCVAFCLLGAGPVDSGVTQTPKHLITATGQRVTLRCSPRSGDLS
VYWYQQSLDQGLQFLIQYYNGEERAKGNILERFSAQQFPDLHSELNLSSL
ELGDSALYFCASSPGGIGYEQYFGPGTRLTVTE

SEQ ID NO. 22 shows the amino acid sequence of the constant region of the T-cell receptor alpha-chain, which comprises murine constant domains (murine alpha)

IQNPEPAVYQLKDPRSQDSTLCLFTDFDSQINVPKTMESGTFITDKCVLD
MKAMDSKSNGAIAWSNQTSFTCQDIFKETNATYPSSDVPCDATLTEKSFE
TDMNLNFQNLSVMGLRILLLKVAGFNLLMTLRLWSS

SEQ ID NO. 23 shows the amino acid sequence of the constant region of the T-cell receptor beta-chain, which comprises murine constant domains (murine beta)

EDLRNVTPPKVSLFEPSKAEIANKQKATLVCLARGFFPDHVELSWWVNGK
EVHSGVCTDPQAYKESNYSYCLSSRLRVSATFWHNPRNHFRCQVQFHGLS
EEDKWPEGSPKPVTQNISAEAWGRADCGITSASYHQGVLSATILYEILLG
KATLYAVLVSGLVLMAMVKKKNS

SEQ ID NO. 5 shows the amino acid sequence of the linker or hinge region which links the beta chain and alpha chain:

GSGATNFSLLKQAGDVEENPGP

SEQ ID NO. 24 shows the full length amino acid sequence of the T-cell receptor alpha-chain consisting of SEQ ID NO. 18 and 22 (wherein SEQ ID NO. 18 is underlined):

MKSLRVLLVILWLQLSWVWSQQKEVEQNSGPLSVPEGAIASLNCTYSDR
GSQSFFWYRQYSGKSPELIMFIYSNGDKEDGRFTAQLNKASQYVSLLIR
DSQPSDSATYLCAAKMKTSYDKVIFGPGTSLSVIPIQNPEPAVYQLKDP
RSQDSTLCLFTDFDSQINVPKTMESGTFITDKCVLDMKAMDSKSNGAIA
WSNQTSFTCQDIFKETNATYPSSDVPCDATLTEKSFETDMNLNFQNLSV
MGLRILLLKVAGFNLLMTLRLWSS*

SEQ ID NO. 25 shows the full length amino acid sequence of the T-cell receptor alpha-chain consisting of SEQ ID NO. 19 and 22 (wherein SEQ ID NO. 19 is underlined):

MKSLRVLLVILWLQLSWVWSQQKEVEQNSGPLSVPEGAIASLNCTYSDR
GSQSFFWYRQYSGKSPELIMFIYSNGDKEDGRFTAQLNKASQYVSLLIR
DSQPSDSATYLCAGYSGGGADGLTFGKGTHLIIQPEDLRNVTPXIQNPE
PAVYQLKDPRSQDSTLCLFTDFDSQINVPKTMESGTFITDKCVLDMKAM
DSKSNGAIAWSNQTSFTCQDIFKETNATYPSSDVPCDATLTEKSFETDM
NLNFQNLSVMGLRILLLKVAGFNLLMTLRLWSS*

SEQ ID NO. 26 shows the full length amino acid sequence of the T-cell receptor alpha-chain consisting of SEQ ID NO. 20 and 22 (wherein SEQ ID NO. 20 is underlined):

MTSIRAVFIFLWLQLDLVNGENVEQHPSTLSVQEGDSAVIKCTYSDSAS
NYFPWYKQELGKRPQLIIDIRSNVGEKKDQRIAVTLNKTAKHFSLHITE
TQPEDSAVYFCAASNGQGAQKLVFGQGTRLTINIQNPEPAVYQLKDPRS
QDSTLCLFTDFDSQINVPKTMESGTFITDKCVLDMKAMDSKSNGAIAWS
NQTSFTCQDIFKETNATYPSSDVPCDATLTEKSFETDMNLNFQNLSVMG
LRILLLKVAGFNLLMTLRLWSS*

SEQ ID NO. 27 shows the full length amino acid sequence of the T-cell receptor beta-chain (wherein SEQ ID NO. 21 is underlined):

MGFRLLCCVAFCLLGAGPVDSGVTQTPKHLITATGQRVTLRCSPRSGDL
SVYWYQQSLDQGLQFLIQYYNGEERAKGNILERFSAQQFPDLHSELNLS
SLELGDSALYFCASSPGGIGYEQYFGPGTRLTVTEEDLRNVTPPKVSLF
EPSKAEIANKQKATLVCLARGFFPDHVELSWWVNGKEVHSGVCTDPQAY
KESNYSYCLSSRLRVSATFWHNPRNHFRCQVQFHGLSEEDKWPEGSPKP
VTQNISAEAWGRADCGITSASYHQGVLSATILYEILLGKATLYAVLVSG
LVLMAMVKKKNS

SEQ ID NO. 28 shows the full length amino acid sequence of one T-cell receptor, including beta chain, linker and the alpha chain of SEQ ID NO. 24:

MGFRLLCCVAFCLLGAGPVDSGVTQTPKHLITATGQRVTLRCSPRSGDL
SVYWYQQSLDQGLQFLIQYYNGEERAKGNILERFSAQQFPDLHSELNLS
SLELGDSALYFCASSPGGIGYEQYFGPGTRLTVTEEDLRNVTPPKVSLF
EPSKAEIANKQKATLVCLARGFFPDHVELSWWVNGKEVHSGVCTDPQAY
KESNYSYCLSSRLRVSATFWHNPRNHFRCQVQFHGLSEEDKWPEGSPKP
VTQNISAEAWGRADCGITSASYHQGVLSATILYEILLGKATLYAVLVSG
LVLMAMVKKKNSGSGATNFSLLKQAGDVEENPGPMKSLRVLLVILWLQL
SWVWSQQKEVEQNSGPLSVPEGAIASLNCTYSDRGSQSFFWYRQYSGKS
PELIMFIYSNGDKEDGRFTAQLNKASQYVSLLIRDSQPSDSATYLCAAK
MKTSYDKVIFGPGTSLSVIPIQNPEPAVYQLKDPRSQDSTLCLFTDFDS
QINVPKTMESGTFITDKCVLDMKAMDSKSNGAIAWSNQTSFTCQDIFKE
TNATYPSSDVPCDATLTEKSFETDMNLNFQNLSVMGLRILLLKVAGFNL
LMTLRLWSS*

SEQ ID NO. 29 shows the full length amino acid sequence of another T-cell receptor, including beta chain, linker and the alpha chain of SEQ ID NO. 25:

MGFRLLCCVAFCLLGAGPVDSGVTQTPKHLITATGQRVTLRCSPRSGDL
SVYWYQQSLDQGLQFLIQYYNGEERAKGNILERFSAQQFPDLHSELNLS
SLELGDSALYFCASSPGGIGYEQYFGPGTRLTVTEEDLRNVTPPKVSLF
EPSKAEIANKQKATLVCLARGFFPDHVELSWWVNGKEVHSGVCTDPQAY
KESNYSYCLSSRLRVSATFWHNPRNHFRCQVQFHGLSEEDKWPEGSPKP
VTQNISAEAWGRADCGITSASYHQGVLSATILYEILLGKATLYAVLVSG
LVLMAMVKKKNSGSGATNFSLLKQAGDVEENPGPMKSLRVLLVILWLQL
SWVWSQQKEVEQNSGPLSVPEGAIASLNCTYSDRGSQSFFWYRQYSGKS
PELIMFIYSNGDKEDGRFTAQLNKASQYVSLLIRDSQPSDSATYLCAGY
SGGGADGLTFGKGTHLIIQPEDLRNVTPXIQNPEPAVYQLKDPRSQDST
LCLFTDFDSQINVPKTMESGTFITDKCVLDMKAMDSKSNGAIAWSNQTS
FTCQDIFKETNATYPSSDVPCDATLTEKSFETDMNLNFQNLSVMGLRIL
LLKVAGFNLLMTLRLWSS*

SEQ ID NO. 30 shows the full length amino acid sequence of another T-cell receptor, including beta chain, linker and the alpha chain of SEQ ID NO. 26:

MGFRLLCCVAFCLLGAGPVDSGVTQTPKHLITATGQRVTLRCSPRSGDL
SVYWYQQSLDQGLQFLIQYYNGEERAKGNILERFSAQQFPDLHSELNLS
SLELGDSALYFCASSPGGIGYEQYFGPGTRLTVTEEDLRNVTPPKVSLF
EPSKAEIANKQKATLVCLARGFFPDHVELSWWVNGKEVHSGVCTDPQAY
KESNYSYCLSSRLRVSATFWHNPRNHFRCQVQFHGLSEEDKWPEGSPKP
VTQNISAEAWGRADCGITSASYHQGVLSATILYEILLGKATLYAVLVSG
LVLMAMVKKKNSGSGATNFSLLKQAGDVEENPGPMTSIRAVFIFLWLQL
DLVNGENVEQHPSTLSVQEGDSAVIKCTYSDSASNYFPWYKQELGKRPQ
LIIDIRSNVGEKKDQRIAVTLNKTAKHFSLHITETQPEDSAVYFCAASN
GQGAQKLVFGQGTRLTINIQNPEPAVYQLKDPRSQDSTLCLFTDFDSQI
NVPKTMESGTFITDKCVLDMKAMDSKSNGAIAWSNQTSFTCQDIFKETN
ATYPSSDVPCDATLTEKSFETDMNLNFQNLSVMGLRILLLKVAGFNLLM
TLRLWSS*

Soluble TCR (sTCR) Constructs

As discussed above, the present invention provides soluble T-cell receptor (sTCR) constructs comprising the T-cell receptors/chains of the present invention.

Said soluble T-cell receptor (sTCR) constructs comprise

(1) at least one of
T-cell receptor α-chain(s) as defined herein,
T-cell receptor β-chain(s) as defined herein,
   that are preferably covalently linked to each other to form TCR heterodimers or multimers, and
   (2) fusion component(s)
preferably selected from
   Fc receptors and/or Fc domains,
   cytokines, such as IL-2 or IL-15,
   toxins, such as exotoxins, e.g. pseudomonas exotoxin,
   antibodies, such as anti-CD3, anti-CD28, anti-CD5, anti-CD16 or anti-CD56 antibodies,
   or combinations thereof,
      wherein the at least one T-cell receptor chain (1) is bound to the fusion component(s) (2),

Preferably, the soluble T-cell receptor comprises furthermore labels, such as radionuclides, gold (particles), fluorophors (such as fluorescein), which are preferably covalently attached/coupled to the soluble TCR, such as to the fusion component(s) (2).

The soluble TCRs can comprise different of the fusion components (2), such as cytokines and Fc domains.

TCR-alpha- and TCR-beta-chains can be solubilized by chemical modifications and linked covalently to each other and to other molecules such as Fc-receptors, cytokines and toxins (as described in Boulter et al, 2005; Lunde et al 2010).

These soluble TCR constructs are suitable to be directly used for the detection and destruction of liver cancer cells, such as HCC cells, without the need of T-cells. This can be achieved by application of soluble T-cell receptor constructs to patients by intravenous, subcutaneous or intramuscular infusion or injections, respectively, or by application to the mucosa of the respiratory tract by inhalation or by spraying.

In one embodiment, the soluble TCR constructs of the present invention are used as recombinant proteins.

Such recombinant proteins are expressed, preferably in E. coli or mammalian cells, and then purified before further use or application.

The present invention provides the expressed and purified soluble TCR constructs.

### Chimeric TCRs

As discussed above, the present invention provides chimeric T-cell receptors comprising at least one of
the T-cell receptor α-chain as defined herein,
the T-cell receptor β-chain as defined herein, or
the T-cell receptor of the present invention,
   wherein the TCR α-chain and/or the TCR β-chain is/are fused to CD3-zeta chain(s) and/or other TCR stimulation domains, such as the intracellular CD28, CD137 or CD134 domain, /more intracellular signaling domains of CD28, CD137 or CD134, preferably via a linker.

In one embodiment, a chimeric TCR of the invention comprises the T-cell receptor α-chain of the invention and the T-cell receptor β-chain of the invention, which are fused to each other via a linker and which are furthermore fused to CD3-zeta chain(s).

Preferred CD3-zeta chain(s) are the zeta-chain(s) of the human CD3 complex of the T-cell receptor. Preferred CD28, CD137 and CD134 signalling domains are of human origin.

The chimeric TCRs can comprise further components.

Chimeric T-cell receptors and chimeric antigen receptors (CAR) are known in the art. See, for example, Han et al. 2013.

### Bi-Specific Antibodies

As discussed above, the present invention provides bi-specific antibodies comprising

(a) the T-cell receptor α- and β-chain(s) of the TCR defined herein,
which are linked with each other
and fused, preferably via a linker, to
   (b) an antibody or a single chain antibody fragment (scFv) which is directed against an antigen or epitope on the surface of lymphocytes.

In one embodiment, the antibody or scFv is/are directed against the CD3 or CD5 receptor of T cells.

In this embodiment, the bi-specific antibody will bind to T cells and redirect them to GPC3-presenting tumor cells.

In another embodiment, the antibody or scFv is/are directed against the co-stimulatory CD28 receptor on T cells.

In this embodiment, the bi-specific antibody will bind to and co-activate T cells and direct them to GPC3-presenting tumor cells.

In another embodiment, antibodies or scFV against CD3 or CD5 and CD28 are combined in tetravalent antibody constructs.

In one embodiment, the antibody or scFv is/are directed against the CD56 receptor of NK cells.

In this embodiment, the bi-specific antibody will bind to NK cells and redirect them to GPC3-presenting tumor cells.

In another embodiment, the antibody or scFv is/are directed against the activating receptor CD16 on NK cells.

In this embodiment, the bi-specific antibody will bind to and activate NK cells and direct them to GPC3-presenting tumor cells.

In another embodiment, antibodies or scFV against CD56 and CD16 are combined in tetravalent antibody constructs.

For example, a bi-specific antibody can be a bi-specific T-cell engager (BiTE) which is a class of artificial bispecific monoclonal antibodies that are investigated for the use as anti-cancer drugs. They direct a host's immune system, more specifically the T cells' cytotoxic activity, against cancer cells. BiTE is a registered trademark of Micromet AG.^{[}BiTEs are fusion proteins consisting of two single-chain variable fragments (scFvs) of different antibodies, or amino acid sequences from four different genes, on a single peptide chain of about 55 kilodaltons. One of the scFvs binds to T cells such as via the CD3 receptor or to CD56 for NK cell activation, and the other to a tumor cell via a tumor specific molecule (see e.g. Csoka et al., 1996).

Preferably, the bi-specific antibodies of the present invention are used as recombinant proteins.

Such recombinant proteins are expressed, preferably in E. coli or mammalian cells, and then purified before further use or application.

The present invention provides the expressed and purified bi-specific antibodies.

### Nucleic Acids, Expression Constructs and Cells Comprising the GPC3-Specific TCR

As discussed above, the present invention provides a nucleic acid encoding the T-cell receptor according to the present invention.

### —Nucleic Acids for TCR Specific for GPC3 Peptide 367

Preferably, the nucleic acid comprises

the nucleic acid encoding for the amino acid sequence of SEQ ID NO. 1 (variable alpha) and/or SEQ ID NO. 2 (variable beta)
or the nucleotide sequence of SEQ ID NO. 9 (variable alpha) and/or SEQ ID NO. 10 (variable beta)
or their complementary sequence(s)
or sequence(s) that have at least 80% identity to the nucleotide sequence of SEQ ID NO. 9 or 10, more preferably at least 85% identity, more preferably 90 or 95 or 99% identity.

SEQ ID NO. 9 shows the nucleotide sequence of the variable region of the T-cell receptor α-chain:

ATGCTGCTGGAACTGATCCCTCTGCTGGGCATCCACTTCGTGCTGAGAA
CCGCCAGAGCCCAGAGCGTGACCCAGCCCGATATCCACATCACCGTGTC
TGAGGGCGCCAGCCTGGAACTGCGGTGCAACTACAGCTACGGCGCCACC
CCCTACCTGTTTTGGTACGTGCAGAGCCCCGGACAGGGCCTGCAGCTGC
TGCTGAAGTACTTTAGCGGCGACACCCTGGTGCAGGGCATCAAGGGATT
CGAGGCCGAGTTCAAGCGGAGCCAGAGCAGCTTCAACCTGCGGAAGCCC
TCCGTGCATTGGAGCGACGCCGCCGAGTACTTTTGTGCCGTGGGCGTGC
TGAACTTCGGCAACGAGAAGCTGACCTTTGGCACCGGCACCCGGCTGAC
CATCATCCCT

SEQ ID NO. 10 shows the nucleotide sequence of the variable region of the T-cell receptor β-chain:

ATGCTGTGTTCTCTGCTGGCTCTGCTGCTGGGCACCTTCTTTGGAGTG
CGGAGCCAGACCATCCACCAGTGGCCTGCTACACTGGTGCAGCCTGTG
GGCAGCCCTCTGAGCCTGGAATGTACCGTGGAAGGCACCAGCAACCCC
AACCTGTACTGGTACAGACAGGCCGCTGGCAGAGGCCTGCAGCTGCTG
TTTTACAGCGTGGGCATCGGCCAGATCAGCAGCGAGGTGCCCCAGAAT
CTGAGCGCCAGCAGACCCCAGGACCGGCAGTTTATCCTGAGCAGCAAG
AAGCTGCTGCTGAGCGACAGCGGCTTCTACCTGTGCGCTTGGAGCGAG
GACACCGCCTACTACGGCTACACCTTTGGCAGCGGCACCCGGCTGACA
GTGGTGGAA

In one embodiment, the nucleic acid comprises

the nucleic acid encoding for the amino acid sequence of SEQ ID NO. 6 (variable alpha+murine alpha) and/or SEQ ID NO. 7 (variable beta+murine beta)
the nucleotide sequence of SEQ ID NO. 11 (variable alpha+murine alpha) and/or SEQ ID NO. 12 (variable beta+murine beta)
or their complementary sequence(s)
or sequence(s) that have at least 80% identity to the nucleotide sequence of SEQ ID NO. 11 or 12, more preferably at least 85% identity, more preferably 90 or 95 or 99% identity.

SEQ ID NO. 11 shows the full length nucleotide sequence of the T-cell receptor alpha-chain (underlined is the variable region/SEQ ID NO. 9):

ATGCTGCTGGAACTGATCCCTCTGCTGGGCATCCACTTCGTGCTGAGA
ACCGCCAGAGCCCAGAGCGTGACCCAGCCCGATATCCACATCACCGTG
TCTGAGGGCGCCAGCCTGGAACTGCGGTGCAACTACAGCTACGGCGCC
ACCCCCTACCTGTTTTGGTACGTGCAGAGCCCCGGACAGGGCCTGCAG
CTGCTGCTGAAGTACTTTAGCGGCGACACCCTGGTGCAGGGCATCAAG
GGATTCGAGGCCGAGTTCAAGCGGAGCCAGAGCAGCTTCAACCTGCGG
AAGCCCTCCGTGCATTGGAGCGACGCCGCCGAGTACTTTTGTGCCGTG
GGCGTGCTGAACTTCGGCAACGAGAAGCTGACCTTTGGCACCGGCACC
CGGCTGACCATCATCCCTATCCAGAACCCCGAGCCCGCCGTGTACCAG
CTGAAGGACCCCAGAAGCCAGGACAGCACCCTGTGTCTGTTCACCGAC
TTCGACAGCCAGATCAACGTGCCCAAGACCATGGAAAGCGGCACCTTC
ATCACCGATAAGTGCGTGCTGGACATGAAGGCCATGGACAGCAAGAGC
AACGGCGCCATTGCCTGGTCCAACCAGACCAGCTTCACATGCCAGGAC
ATCTTCAAAGAGACAAACGCCACCTACCCTTCCAGCGACGTGCCCTGT
GACGCCACCCTGACCGAGAAGTCCTTCGAGACAGACATGAATCTGAAT
TTCCAGAACCTGAGCGTGATGGGCCTGCGGATCCTGCTGCTGAAAGTG
GCCGGCTTCAACCTGCTGATGACCCTGCGGCTGTGGTCCAGCTGA

SEQ ID NO. 12 shows the full length nucleotide sequence of the T-cell receptor beta-chain (underlined is the variable region/SEQ ID NO. 10):

ATGCTGTGTTCTCTGCTGGCTCTGCTGCTGGGCACCTTCTTTGGAGTG
CGGAGCCAGACCATCCACCAGTGGCCTGCTACACTGGTGCAGCCTGTG
GGCAGCCCTCTGAGCCTGGAATGTACCGTGGAAGGCACCAGCAACCCC
AACCTGTACTGGTACAGACAGGCCGCTGGCAGAGGCCTGCAGCTGCTG
TTTTACAGCGTGGGCATCGGCCAGATCAGCAGCGAGGTGCCCCAGAAT
CTGAGCGCCAGCAGACCCCAGGACCGGCAGTTTATCCTGAGCAGCAAG
AAGCTGCTGCTGAGCGACAGCGGCTTCTACCTGTGCGCTTGGAGCGAG
GACACCGCCTACTACGGCTACACCTTTGGCAGCGGCACCCGGCTGACA
GTGGTGGAAGAAGATCTGAGGAACGTGACCCCCCCCAAGGTGTCCCTG
TTCGAGCCCAGCAAGGCCGAGATCGCCAACAAGCAGAAAGCCACCCTC
GTGTGCCTGGCCAGAGGCTTCTTCCCCGACCACGTGGAACTGTCCTGG
TGGGTCAACGGCAAAGAGGTGCACAGCGGAGTCTGCACCGACCCCCAG
GCTTACAAAGAGAGCAACTACAGCTACTGCCTGTCCAGCAGACTGCGG
GTGTCCGCTACCTTCTGGCACAACCCCCGGAACCACTTCAGATGCCAG
GTGCAGTTCCACGGCCTGAGCGAAGAGGACAAGTGGCCCGAGGGCAGC
CCCAAGCCCGTGACCCAGAATATCAGCGCCGAGGCCTGGGGCAGAGCC
GATTGTGGCATCACCAGCGCCAGCTACCACCAGGGGGTGCTGAGCGCC
ACCATCCTGTACGAGATCCTGCTGGGCAAGGCCACCCTGTACGCCGTG
CTGGTGTCCGGACTGGTGCTGATGGCCATGGTCAAGAAGAAGAACAGC

In one embodiment, the nucleic acid comprises

the nucleic acid encoding for the amino acid sequence of SEQ ID NO. 8 (aa sequence of the entire TCR P1-1)
the nucleotide sequence of SEQ ID NO. 13 (entire TCR P1-1)
or their complementary sequence(s)
or sequence(s) that have at least 80% identity to the nucleotide sequence of SEQ ID NO. 13, more preferably at least 85% identity, more preferably 90 or 95 or 99% identity.

SEQ ID NO. 13 shows the nucleotide sequence of the T-cell receptor (underlined is the linker or hinge region):

ATGCTGTGTTCTCTGCTGGCTCTGCTGCTGGGCACCTTCTTTGGAGTG
CGGAGCCAGACCATCCACCAGTGGCCTGCTACACTGGTGCAGCCTGTG
GGCAGCCCTCTGAGCCTGGAATGTACCGTGGAAGGCACCAGCAACCCC
AACCTGTACTGGTACAGACAGGCCGCTGGCAGAGGCCTGCAGCTGCTG
TTTTACAGCGTGGGCATCGGCCAGATCAGCAGCGAGGTGCCCCAGAAT
CTGAGCGCCAGCAGACCCCAGGACCGGCAGTTTATCCTGAGCAGCAAG
AAGCTGCTGCTGAGCGACAGCGGCTTCTACCTGTGCGCTTGGAGCGAG
GACACCGCCTACTACGGCTACACCTTTGGCAGCGGCACCCGGCTGACA
GTGGTGGAAGAAGATCTGAGGAACGTGACCCCCCCCAAGGTGTCCCTG
TTCGAGCCCAGCAAGGCCGAGATCGCCAACAAGCAGAAAGCCACCCTC
GTGTGCCTGGCCAGAGGCTTCTTCCCCGACCACGTGGAACTGTCCTGG
TGGGTCAACGGCAAAGAGGTGCACAGCGGAGTCTGCACCGACCCCCAG
GCTTACAAAGAGAGCAACTACAGCTACTGCCTGTCCAGCAGACTGCGG
GTGTCCGCTACCTTCTGGCACAACCCCCGGAACCACTTCAGATGCCAG
GTGCAGTTCCACGGCCTGAGCGAAGAGGACAAGTGGCCCGAGGGCAGC
CCCAAGCCCGTGACCCAGAATATCAGCGCCGAGGCCTGGGGCAGAGCC
GATTGTGGCATCACCAGCGCCAGCTACCACCAGGGGGTGCTGAGCGCC
ACCATCCTGTACGAGATCCTGCTGGGCAAGGCCACCCTGTACGCCGTG
CTGGTGTCCGGACTGGTGCTGATGGCCATGGTCAAGAAGAAGAACAGC
GGCAGCGGCGCCACCAACTTCAGCCTGCTGAAGCAGGCCGGCGACGTG
GAAGAGAACCCCGGGCCCATGCTGCTGGAACTGATCCCTCTGCTGGGC
ATCCACTTCGTGCTGAGAACCGCCAGAGCCCAGAGCGTGACCCAGCCC
GATATCCACATCACCGTGTCTGAGGGCGCCAGCCTGGAACTGCGGTGC
AACTACAGCTACGGCGCCACCCCCTACCTGTTTTGGTACGTGCAGAGC
CCCGGACAGGGCCTGCAGCTGCTGCTGAAGTACTTTAGCGGCGACACC
CTGGTGCAGGGCATCAAGGGATTCGAGGCCGAGTTCAAGCGGAGCCAG
AGCAGCTTCAACCTGCGGAAGCCCTCCGTGCATTGGAGCGACGCCGCC
GAGTACTTTTGTGCCGTGGGCGTGCTGAACTTCGGCAACGAGAAGCTG
ACCTTTGGCACCGGCACCCGGCTGACCATCATCCCTATCCAGAACCCC
GAGCCCGCCGTGTACCAGCTGAAGGACCCCAGAAGCCAGGACAGCACC
CTGTGTCTGTTCACCGACTTCGACAGCCAGATCAACGTGCCCAAGACC
ATGGAAAGCGGCACCTTCATCACCGATAAGTGCGTGCTGGACATGAAG
GCCATGGACAGCAAGAGCAACGGCGCCATTGCCTGGTCCAACCAGACC
AGCTTCACATGCCAGGACATCTTCAAAGAGACAAACGCCACCTACCCT
TCCAGCGACGTGCCCTGTGACGCCACCCTGACCGAGAAGTCCTTCGAG
ACAGACATGAATCTGAATTTCCAGAACCTGAGCGTGATGGGCCTGCGG
ATCCTGCTGCTGAAAGTGGCCGGCTTCAACCTGCTGATGACCCTGCGG
CTGTGGTCCAGCTGA

The nucleotide sequences of SEQ ID NO. 9 to 13 are derived from cDNA or are DNA.

The nucleotide sequences of SEQ ID NO. 9 to 13 are codon-optimized.

### —Nucleic Acids for TCR Specific for GPC3 Peptide 326

Preferably, the nucleic acid comprises

the nucleic acid encoding for the amino acid sequence of one of SEQ ID NOs. 18-20 (3× variable alpha) and/or SEQ ID NO. 21 (variable beta)
or the nucleotide sequence of one of SEQ ID NOs. 31-33 (3× variable alpha) and/or SEQ ID NO. 34 (variable beta)
or their complementary sequence(s)
or sequence(s) that have at least 80% identity to the nucleotide sequence of one of SEQ ID NOs. 31-34, more preferably at least 85% identity, more preferably 90 or 95 or 99% identity.

SEQ ID NO. 31 shows the nucleotide sequence of the variable region of one T-cell receptor α-chain:

ATGAAATCCTTGAGAGTTTTACTAGTGATCCTGTGGCTTCAGTTGAGCT
GGGTTTGGAGCCAACAGAAGGAGGTGGAGCAGAATTCTGGACCCCTCAG
TGTTCCAGAGGGAGCCATTGCCTCTCTCAACTGCACTTACAGTGACCGA
GGTTCCCAGTCCTTCTTCTGGTACAGACAATATTCTGGGAAAAGCCCTG
AGTTGATAATGTTCATATACTCCAATGGTGACAAAGAAGATGGAAGGTT
TACAGCACAGCTCAATAAAGCCAGCCAGTATGTTTCTCTGCTCATCAGA
GACTCCCAGCCCAGTGATTCAGCCACCTACCTCTGTGCCGCGAAAATGA
AAACCTCCTACGACAAGGTGATATTTGGGCCAGGGACAAGCTTATCAGT
CATTCCA

SEQ ID NO. 32 shows the nucleotide sequence of the variable region of another T-cell receptor α-chain

ATGAAATCCTTGAGAGTTTTACTAGTGATCCTGTGGCTTCAGTTGAGCT
GGGTTTGGAGCCAACAGAAGGAGGTGGAGCAGAATTCTGGACCCCTCAG
TGTTCCAGAGGGAGCCATTGCCTCTCTCAACTGCACTTACAGTGACCGA
GGTTCCCAGTCCTTCTTCTGGTACAGACAATATTCTGGGAAAAGCCCTG
AGTTGATAATGTTCATATACTCCAATGGTGACAAAGAAGATGGAAGGTT
TACAGCACAGCTCAATAAAGCCAGCCAGTATGTTTCTCTGCTCATCAGA
GACTCCCAGCCCAGTGATTCAGCCACCTACCTCTGTGCCGGGTATTCAG
GAGGAGGTGCTGACGGACTCACCTTTGGCAAAGGGACTCATCTAATCAT
CCAGCCCGAAGATCTGAGGAACGTGACCCCCCC

SEQ ID NO. 33 shows the nucleotide sequence of the variable region of another T-cell receptor α-chain:

ATGACATCCATTCGAGCTGTATTTATATTCCTGTGGCTGCAGCTGGAC
TTGGTGAATGGAGAGAATGTGGAGCAGCATCCTTCAACCCTGAGTGTC
CAGGAGGGAGACAGCGCTGTTATCAAGTGTACTTATTCAGACAGTGCC
TCAAACTACTTCCCTTGGTATAAGCAAGAACTTGGAAAAAGACCTCAG
CTTATTATAGACATTCGTTCAAATGTGGGCGAAAAGAAAGACCAACGA
ATTGCTGTTACATTGAACAAGACAGCCAAACATTTCTCCCTGCACATC
ACAGAGACCCAACCTGAAGACTCGGCTGTCTACTTCTGTGCAGCAAGT
AATGGGCAGGGAGCCCAGAAGCTGGTATTTGGCCAAGGAACCAGGCTG
ACTATCAAC

SEQ ID NO. 34 shows the nucleotide sequence of the variable region of the T-cell receptor β-chain:

ATGGGCTTCAGGCTCCTCTGCTGTGTGGCCTTTTGTCTCCTGGGAGCAG
GCCCAGTGGATTCTGGAGTCACACAAACCCCAAAGCACCTGATCACAGC
AACTGGACAGCGAGTGACGCTGAGATGCTCCCCTAGGTCTGGAGACCTC
TCTGTGTACTGGTACCAACAGAGCCTGGACCAGGGCCTCCAGTTCCTCA
TTCAGTATTATAATGGAGAAGAGAGAGCAAAAGGAAACATTCTTGAACG
ATTCTCCGCACAACAGTTCCCTGACTTGCACTCTGAACTAAACCTGAGC
TCTCTGGAGCTGGGGGACTCAGCTTTGTATTTCTGTGCCAGCAGCCCCG
GGGGGATCGGGTACGAGCAGTACTTCGGGCCGGGCACCAGGCTCACGGT
CACAGAG

In one embodiment, the nucleic acid comprises

the nucleic acid encoding for the amino acid sequence of one of SEQ ID NOs. 24-26 (variable alpha+murine alpha) and/or SEQ ID NO. 27 (variable beta+murine beta)
the nucleotide sequence of one of SEQ ID NOs. 35-37 (variable alpha+murine alpha)
and/or SEQ ID NO. 38 (variable beta+murine beta)
or their complementary sequence(s)
or sequence(s) that have at least 80% identity to the nucleotide sequence of one of SEQ ID NOs. 35-38, more preferably at least 85% identity, more preferably 90 or 95 or 99% identity.

SEQ ID NO. 35 shows the full length nucleotide sequence of one T-cell receptor alpha-chain (underlined is the variable region/SEQ ID NO. 31):

ATGAAATCCTTGAGAGTTTTACTAGTGATCCTGTGGCTTCAGTTGAGC
TGGGTTTGGAGCCAACAGAAGGAGGTGGAGCAGAATTCTGGACCCCTC
AGTGTTCCAGAGGGAGCCATTGCCTCTCTCAACTGCACTTACAGTGAC
CGAGGTTCCCAGTCCTTCTTCTGGTACAGACAATATTCTGGGAAAAGC
CCTGAGTTGATAATGTTCATATACTCCAATGGTGACAAAGAAGATGGA
AGGTTTACAGCACAGCTCAATAAAGCCAGCCAGTATGTTTCTCTGCTC
ATCAGAGACTCCCAGCCCAGTGATTCAGCCACCTACCTCTGTGCCGCG
AAAATGAAAACCTCCTACGACAAGGTGATATTTGGGCCAGGGACAAGC
TTATCAGTCATTCCAATCCAGAACCCCGAGCCCGCCGTGTACCAGCTG
AAGGACCCCAGAAGCCAGGACAGCACCCTGTGTCTGTTCACCGACTTC
GACAGCCAGATCAACGTGCCCAAGACCATGGAAAGCGGCACCTTCATC
ACCGATAAGTGCGTGCTGGACATGAAGGCCATGGACAGCAAGAGCAAC
GGCGCCATTGCCTGGTCCAACCAGACCAGCTTCACATGCCAGGACATC
TTCAAAGAGACAAACGCCACCTACCCTTCCAGCGACGTGCCCTGTGAC
GCCACCCTGACCGAGAAGTCCTTCGAGACAGACATGAATCTGAATTTC
CAGAACCTGAGCGTGATGGGCCTGCGGATCCTGCTGCTGAAAGTGGCC
GGCTTCAACCTGCTGATGACCCTGCGGCTGTGGTCCAGCTGA

SEQ ID NO. 36 shows the full length nucleotide sequence of another T-cell receptor alpha-chain (underlined is the variable region/SEQ ID NO. 32):

ATGAAATCCTTGAGAGTTTTACTAGTGATCCTGTGGCTTCAGTTGAGC
TGGGTTTGGAGCCAACAGAAGGAGGTGGAGCAGAATTCTGGACCCCTC
AGTGTTCCAGAGGGAGCCATTGCCTCTCTCAACTGCACTTACAGTGAC
CGAGGTTCCCAGTCCTTCTTCTGGTACAGACAATATTCTGGGAAAAGC
CCTGAGTTGATAATGTTCATATACTCCAATGGTGACAAAGAAGATGGA
AGGTTTACAGCACAGCTCAATAAAGCCAGCCAGTATGTTTCTCTGCTC
ATCAGAGACTCCCAGCCCAGTGATTCAGCCACCTACCTCTGTGCCGGG
TATTCAGGAGGAGGTGCTGACGGACTCACCTTTGGCAAAGGGACTCAT
CTAATCATCCAGCCCGAAGATCTGAGGAACGTGACCCCCCCATCCAGA
ACCCCGAGCCCGCCGTGTACCAGCTGAAGGACCCCAGAAGCCAGGACA
GCACCCTGTGTCTGTTCACCGACTTCGACAGCCAGATCAACGTGCCCA
AGACCATGGAAAGCGGCACCTTCATCACCGATAAGTGCGTGCTGGACA
TGAAGGCCATGGACAGCAAGAGCAACGGCGCCATTGCCTGGTCCAACC
AGACCAGCTTCACATGCCAGGACATCTTCAAAGAGACAAACGCCACCT
ACCCTTCCAGCGACGTGCCCTGTGACGCCACCCTGACCGAGAAGTCCT
TCGAGACAGACATGAATCTGAATTTCCAGAACCTGAGCGTGATGGGCC
TGCGGATCCTGCTGCTGAAAGTGGCCGGCTTCAACCTGCTGATGACCC
TGCGGCTGTGGTCCAGCTGA

SEQ ID NO. 37 shows the full length nucleotide sequence of another T-cell receptor alpha-chain (underlined is the variable region/SEQ ID NO. 33):

ATGACATCCATTCGAGCTGTATTTATATTCCTGTGGCTGCAGCTGGAC
TTGGTGAATGGAGAGAATGTGGAGCAGCATCCTTCAACCCTGAGTGTC
CAGGAGGGAGACAGCGCTGTTATCAAGTGTACTTATTCAGACAGTGCC
TCAAACTACTTCCCTTGGTATAAGCAAGAACTTGGAAAAAGACCTCAG
CTTATTATAGACATTCGTTCAAATGTGGGCGAAAAGAAAGACCAACGA
ATTGCTGTTACATTGAACAAGACAGCCAAACATTTCTCCCTGCACATC
ACAGAGACCCAACCTGAAGACTCGGCTGTCTACTTCTGTGCAGCAAGT
AATGGGCAGGGAGCCCAGAAGCTGGTATTTGGCCAAGGAACCAGGCTG
ACTATCAACATCCAGAACCCCGAGCCCGCCGTGTACCAGCTGAAGGAC
CCCAGAAGCCAGGACAGCACCCTGTGTCTGTTCACCGACTTCGACAGC
CAGATCAACGTGCCCAAGACCATGGAAAGCGGCACCTTCATCACCGAT
AAGTGCGTGCTGGACATGAAGGCCATGGACAGCAAGAGCAACGGCGCC
ATTGCCTGGTCCAACCAGACCAGCTTCACATGCCAGGACATCTTCAAA
GAGACAAACGCCACCTACCCTTCCAGCGACGTGCCCTGTGACGCCACC
CTGACCGAGAAGTCCTTCGAGACAGACATGAATCTGAATTTCCAGAAC
CTGAGCGTGATGGGCCTGCGGATCCTGCTGCTGAAAGTGGCCGGCTTC
AACCTGCTGATGACCCTGCGGCTGTGGTCCAGCTGA

SEQ ID NO. 38 shows the full length nucleotide sequence of the T-cell receptor beta-chain (underlined is the variable region/SEQ ID NO. 34):

ATGGGCTTCAGGCTCCTCTGCTGTGTGGCCTTTTGTCTCCTGGGAGCAG
GCCCAGTGGATTCTGGAGTCACACAAACCCCAAAGCACCTGATCACAGC
AACTGGACAGCGAGTGACGCTGAGATGCTCCCCTAGGTCTGGAGACCTC
TCTGTGTACTGGTACCAACAGAGCCTGGACCAGGGCCTCCAGTTCCTCA
TTCAGTATTATAATGGAGAAGAGAGAGCAAAAGGAAACATTCTTGAACG
ATTCTCCGCACAACAGTTCCCTGACTTGCACTCTGAACTAAACCTGAGC
TCTCTGGAGCTGGGGGACTCAGCTTTGTATTTCTGTGCCAGCAGCCCCG
GGGGGATCGGGTACGAGCAGTACTTCGGGCCGGGCACCAGGCTCACGGT
CACAGAGGAAGATCTGAGGAACGTGACCCCCCCCAAGGTGTCCCTGTTC
GAGCCCAGCAAGGCCGAGATCGCCAACAAGCAGAAAGCCACCCTCGTGT
GCCTGGCCAGAGGCTTCTTCCCCGACCACGTGGAACTGTCCTGGTGGGT
CAACGGCAAAGAGGTGCACAGCGGAGTCTGCACCGACCCCCAGGCTTAC
AAAGAGAGCAACTACAGCTACTGCCTGTCCAGCAGACTGCGGGTGTCCG
CTACCTTCTGGCACAACCCCCGGAACCACTTCAGATGCCAGGTGCAGTT
CCACGGCCTGAGCGAAGAGGACAAGTGGCCCGAGGGCAGCCCCAAGCCC
GTGACCCAGAATATCAGCGCCGAGGCCTGGGGCAGAGCCGATTGTGGCA
TCACCAGCGCCAGCTACCACCAGGGGGTGCTGAGCGCCACCATCCTGTA
CGAGATCCTGCTGGGCAAGGCCACCCTGTACGCCGTGCTGGTGTCCGGA
CTGGTGCTGATGGCCATGGTCAAGAAGAAGAACAGC

In one embodiment, the nucleic acid comprises

the nucleic acid encoding for the amino acid sequence of one of SEQ ID NOs. 28-30 (3× aa sequence of the entire TCR P2-1)
the nucleotide sequence of one of SEQ ID NOs. 39-41 (entire TCR P2-1)
or their complementary sequence(s)
or sequence(s) that have at least 80% identity to the nucleotide sequence of one of SEQ ID NOs. 39-41, more preferably at least 85% identity, more preferably 90 or 95 or 99% identity.

SEQ ID NO. 39 shows the nucleotide sequence of one T-cell receptor (underlined is the linker or hinge region):

ATGGGCTTCAGGCTCCTCTGCTGTGTGGCCTTTTGTCTCCTGGGAGCAG
GCCCAGTGGATTCTGGAGTCACACAAACCCCAAAGCACCTGATCACAGC
AACTGGACAGCGAGTGACGCTGAGATGCTCCCCTAGGTCTGGAGACCTC
TCTGTGTACTGGTACCAACAGAGCCTGGACCAGGGCCTCCAGTTCCTCA
TTCAGTATTATAATGGAGAAGAGAGAGCAAAAGGAAACATTCTTGAACG
ATTCTCCGCACAACAGTTCCCTGACTTGCACTCTGAACTAAACCTGAGC
TCTCTGGAGCTGGGGGACTCAGCTTTGTATTTCTGTGCCAGCAGCCCCG
GGGGGATCGGGTACGAGCAGTACTTCGGGCCGGGCACCAGGCTCACGGT
CACAGAGGAAGATCTGAGGAACGTGACCCCCCCCAAGGTGTCCCTGTTC
GAGCCCAGCAAGGCCGAGATCGCCAACAAGCAGAAAGCCACCCTCGTGT
GCCTGGCCAGAGGCTTCTTCCCCGACCACGTGGAACTGTCCTGGTGGGT
CAACGGCAAAGAGGTGCACAGCGGAGTCTGCACCGACCCCCAGGCTTAC
AAAGAGAGCAACTACAGCTACTGCCTGTCCAGCAGACTGCGGGTGTCCG
CTACCTTCTGGCACAACCCCCGGAACCACTTCAGATGCCAGGTGCAGTT
CCACGGCCTGAGCGAAGAGGACAAGTGGCCCGAGGGCAGCCCCAAGCCC
GTGACCCAGAATATCAGCGCCGAGGCCTGGGGCAGAGCCGATTGTGGCA
TCACCAGCGCCAGCTACCACCAGGGGGTGCTGAGCGCCACCATCCTGTA
CGAGATCCTGCTGGGCAAGGCCACCCTGTACGCCGTGCTGGTGTCCGGA
CTGGTGCTGATGGCCATGGTCAAGAAGAAGAACAGCGGCAGCGGCGCCA
CCAACTTCAGCCTGCTGAAGCAGGCCGGCGACGTGGAAGAGAACCCCGG
GCCCATGAAATCCTTGAGAGTTTTACTAGTGATCCTGTGGCTTCAGTTG
AGCTGGGTTTGGAGCCAACAGAAGGAGGTGGAGCAGAATTCTGGACCCC
TCAGTGTTCCAGAGGGAGCCATTGCCTCTCTCAACTGCACTTACAGTGA
CCGAGGTTCCCAGTCCTTCTTCTGGTACAGACAATATTCTGGGAAAAGC
CCTGAGTTGATAATGTTCATATACTCCAATGGTGACAAAGAAGATGGAA
GGTTTACAGCACAGCTCAATAAAGCCAGCCAGTATGTTTCTCTGCTCAT
CAGAGACTCCCAGCCCAGTGATTCAGCCACCTACCTCTGTGCCGCGAAA
ATGAAAACCTCCTACGACAAGGTGATATTTGGGCCAGGGACAAGCTTAT
CAGTCATTCCAATCCAGAACCCCGAGCCCGCCGTGTACCAGCTGAAGGA
CCCCAGAAGCCAGGACAGCACCCTGTGTCTGTTCACCGACTTCGACAGC
CAGATCAACGTGCCCAAGACCATGGAAAGCGGCACCTTCATCACCGATA
AGTGCGTGCTGGACATGAAGGCCATGGACAGCAAGAGCAACGGCGCCAT
TGCCTGGTCCAACCAGACCAGCTTCACATGCCAGGACATCTTCAAAGAG
ACAAACGCCACCTACCCTTCCAGCGACGTGCCCTGTGACGCCACCCTGA
CCGAGAAGTCCTTCGAGACAGACATGAATCTGAATTTCCAGAACCTGAG
CGTGATGGGCCTGCGGATCCTGCTGCTGAAAGTGGCCGGCTTCAACCTG
CTGATGACCCTGCGGCTGTGGTCCAGCTGA

SEQ ID NO. 40 shows the nucleotide sequence of another T-cell receptor (underlined is the linker or hinge region):

ATGGGCTTCAGGCTCCTCTGCTGTGTGGCCTTTTGTCTCCTGGGAGCAG
GCCCAGTGGATTCTGGAGTCACACAAACCCCAAAGCACCTGATCACAGC
AACTGGACAGCGAGTGACGCTGAGATGCTCCCCTAGGTCTGGAGACCTC
TCTGTGTACTGGTACCAACAGAGCCTGGACCAGGGCCTCCAGTTCCTCA
TTCAGTATTATAATGGAGAAGAGAGAGCAAAAGGAAACATTCTTGAACG
ATTCTCCGCACAACAGTTCCCTGACTTGCACTCTGAACTAAACCTGAGC
TCTCTGGAGCTGGGGGACTCAGCTTTGTATTTCTGTGCCAGCAGCCCCG
GGGGGATCGGGTACGAGCAGTACTTCGGGCCGGGCACCAGGCTCACGGT
CACAGAGGAAGATCTGAGGAACGTGACCCCCCCCAAGGTGTCCCTGTTC
GAGCCCAGCAAGGCCGAGATCGCCAACAAGCAGAAAGCCACCCTCGTGT
GCCTGGCCAGAGGCTTCTTCCCCGACCACGTGGAACTGTCCTGGTGGGT
CAACGGCAAAGAGGTGCACAGCGGAGTCTGCACCGACCCCCAGGCTTAC
AAAGAGAGCAACTACAGCTACTGCCTGTCCAGCAGACTGCGGGTGTCCG
CTACCTTCTGGCACAACCCCCGGAACCACTTCAGATGCCAGGTGCAGTT
CCACGGCCTGAGCGAAGAGGACAAGTGGCCCGAGGGCAGCCCCAAGCCC
GTGACCCAGAATATCAGCGCCGAGGCCTGGGGCAGAGCCGATTGTGGCA
TCACCAGCGCCAGCTACCACCAGGGGGTGCTGAGCGCCACCATCCTGTA
CGAGATCCTGCTGGGCAAGGCCACCCTGTACGCCGTGCTGGTGTCCGGA
CTGGTGCTGATGGCCATGGTCAAGAAGAAGAACAGCGGCAGCGGCGCCA
CCAACTTCAGCCTGCTGAAGCAGGCCGGCGACGTGGAAGAGAACCCCGG
GCCCATGAAATCCTTGAGAGTTTTACTAGTGATCCTGTGGCTTCAGTTG
AGCTGGGTTTGGAGCCAACAGAAGGAGGTGGAGCAGAATTCTGGACCCC
TCAGTGTTCCAGAGGGAGCCATTGCCTCTCTCAACTGCACTTACAGTGA
CCGAGGTTCCCAGTCCTTCTTCTGGTACAGACAATATTCTGGGAAAAGC
CCTGAGTTGATAATGTTCATATACTCCAATGGTGACAAAGAAGATGGAA
GGTTTACAGCACAGCTCAATAAAGCCAGCCAGTATGTTTCTCTGCTCAT
CAGAGACTCCCAGCCCAGTGATTCAGCCACCTACCTCTGTGCCGGGTAT
TCAGGAGGAGGTGCTGACGGACTCACCTTTGGCAAAGGGACTCATCTAA
TCATCCAGCCCGAAGATCTGAGGAACGTGACCCCCCCATCCAGAACCCC
GAGCCCGCCGTGTACCAGCTGAAGGACCCCAGAAGCCAGGACAGCACCC
TGTGTCTGTTCACCGACTTCGACAGCCAGATCAACGTGCCCAAGACCAT
GGAAAGCGGCACCTTCATCACCGATAAGTGCGTGCTGGACATGAAGGCC
ATGGACAGCAAGAGCAACGGCGCCATTGCCTGGTCCAACCAGACCAGCT
TCACATGCCAGGACATCTTCAAAGAGACAAACGCCACCTACCCTTCCAG
CGACGTGCCCTGTGACGCCACCCTGACCGAGAAGTCCTTCGAGACAGAC
ATGAATCTGAATTTCCAGAACCTGAGCGTGATGGGCCTGCGGATCCTGC
TGCTGAAAGTGGCCGGCTTCAACCTGCTGATGACCCTGCGGCTGTGGTC
CAGCTGA

SEQ ID NO. 41 shows the nucleotide sequence of another T-cell receptor (underlined is the linker or hinge region):

ATGGGCTTCAGGCTCCTCTGCTGTGTGGCCTTTTGTCTCCTGGGAGCAG
GCCCAGTGGATTCTGGAGTCACACAAACCCCAAAGCACCTGATCACAGC
AACTGGACAGCGAGTGACGCTGAGATGCTCCCCTAGGTCTGGAGACCTC
TCTGTGTACTGGTACCAACAGAGCCTGGACCAGGGCCTCCAGTTCCTCA
TTCAGTATTATAATGGAGAAGAGAGAGCAAAAGGAAACATTCTTGAACG
ATTCTCCGCACAACAGTTCCCTGACTTGCACTCTGAACTAAACCTGAGC
TCTCTGGAGCTGGGGGACTCAGCTTTGTATTTCTGTGCCAGCAGCCCCG
GGGGGATCGGGTACGAGCAGTACTTCGGGCCGGGCACCAGGCTCACGGT
CACAGAGGAAGATCTGAGGAACGTGACCCCCCCCAAGGTGTCCCTGTTC
GAGCCCAGCAAGGCCGAGATCGCCAACAAGCAGAAAGCCACCCTCGTGT
GCCTGGCCAGAGGCTTCTTCCCCGACCACGTGGAACTGTCCTGGTGGGT
CAACGGCAAAGAGGTGCACAGCGGAGTCTGCACCGACCCCCAGGCTTAC
AAAGAGAGCAACTACAGCTACTGCCTGTCCAGCAGACTGCGGGTGTCCG
CTACCTTCTGGCACAACCCCCGGAACCACTTCAGATGCCAGGTGCAGTT
CCACGGCCTGAGCGAAGAGGACAAGTGGCCCGAGGGCAGCCCCAAGCCC
GTGACCCAGAATATCAGCGCCGAGGCCTGGGGCAGAGCCGATTGTGGCA
TCACCAGCGCCAGCTACCACCAGGGGGTGCTGAGCGCCACCATCCTGTA
CGAGATCCTGCTGGGCAAGGCCACCCTGTACGCCGTGCTGGTGTCCGGA
CTGGTGCTGATGGCCATGGTCAAGAAGAAGAACAGCGGCAGCGGCGCCA
CCAACTTCAGCCTGCTGAAGCAGGCCGGCGACGTGGAAGAGAACCCCGG
GCCCATGACATCCATTCGAGCTGTATTTATATTCCTGTGGCTGCAGCTG
GACTTGGTGAATGGAGAGAATGTGGAGCAGCATCCTTCAACCCTGAGTG
TCCAGGAGGGAGACAGCGCTGTTATCAAGTGTACTTATTCAGACAGTGC
CTCAAACTACTTCCCTTGGTATAAGCAAGAACTTGGAAAAAGACCTCAG
CTTATTATAGACATTCGTTCAAATGTGGGCGAAAAGAAAGACCAACGAA
TTGCTGTTACATTGAACAAGACAGCCAAACATTTCTCCCTGCACATCAC
AGAGACCCAACCTGAAGACTCGGCTGTCTACTTCTGTGCAGCAAGTAAT
GGGCAGGGAGCCCAGAAGCTGGTATTTGGCCAAGGAACCAGGCTGACTA
TCAACATCCAGAACCCCGAGCCCGCCGTGTACCAGCTGAAGGACCCCAG
AAGCCAGGACAGCACCCTGTGTCTGTTCACCGACTTCGACAGCCAGATC
AACGTGCCCAAGACCATGGAAAGCGGCACCTTCATCACCGATAAGTGCG
TGCTGGACATGAAGGCCATGGACAGCAAGAGCAACGGCGCCATTGCCTG
GTCCAACCAGACCAGCTTCACATGCCAGGACATCTTCAAAGAGACAAAC
GCCACCTACCCTTCCAGCGACGTGCCCTGTGACGCCACCCTGACCGAGA
AGTCCTTCGAGACAGACATGAATCTGAATTTCCAGAACCTGAGCGTGAT
GGGCCTGCGGATCCTGCTGCTGAAAGTGGCCGGCTTCAACCTGCTGATG
ACCCTGCGGCTGTGGTCCAGCTGA

The nucleotide sequences of SEQ ID NO. 31 to 41 are derived from cDNA or are DNA.

As discussed above, the present invention provides a nucleic acid encoding the soluble T-cell receptor construct according to the present invention.

As discussed above, the present invention provides a nucleic acid encoding the chimeric T-cell receptor according to the present invention.

As discussed above, the present invention provides a nucleic acid encoding the bi-specific antibody according to the present invention.

The nucleic acids according to this invention comprise DNA (such as dsDNA, ssDNA, cDNA), RNA (such as dsRNA, ssRNA, mRNA), combinations thereof or derivatives (such as PNA) thereof.

Preferably, the nucleic acid sequences of the present invention are codon-optimized for expression in mammalian cells, preferably for expression in human cells. For generation of bi-specific antibodies the nucleic acid sequences can also be codon-optimized for expression in bacteria, yeast or insect cells. Codon-optimization refers to the exchange in a sequence of interest of codons that are generally rare in highly expressed genes of a given species by codons that are generally frequent in highly expressed genes of such species, such codons encoding the same amino acids as the codons that are being exchanged.

Within the scope of this invention are also the nucleotide sequences obtained due to the degeneration of the genetic code of the above nucleotide sequences.

As discussed above, the present invention provides an expression construct for expressing the T-cell receptor according to the present invention in a cell.

As discussed above, the present invention provides an expression construct for expressing the soluble T-cell receptor construct according to the present invention in a cell.

As discussed above, the present invention provides an expression construct for expressing the chimeric T-cell receptor according to the present invention in a cell.

As discussed above, the present invention provides an expression construct for expressing the bi-specific antibody according to the present invention as well as the expressed and purified bi-specific antibody.

Preferably, the expression constructs further comprise promoter and terminator sequences.

An “expression or gene construct” (wherein both terms are used interchangeably throughout this specification) refers to a nucleic acid construct, usually an expression vector or plasmid that is used to introduce a specific gene sequence into a target cell. Once the expression or gene construct is inside the cell, the protein that is encoded by the gene is produced by the cellular transcription and translation machinery. The expression or gene construct is designed to contain respective regulatory sequences that act as enhancer and promoter regions and lead to efficient transcription of the gene carried on the construct, including promoter and terminator sequences. The goal of a well-designed expression or gene construct is the production of large amounts of stable mRNA, and therefore proteins. Alternatively, an RNA is synthesized or in vitro transcribed and introduced directly into the cells for protein expression.

The skilled artisan can select further suitable components of expression or gene constructs.

Examples for suitable expression constructs are viral vectors, such as
retroviral vectors (as described in Verra et al, 2004; Porter et al, 2011), such as MP71 vectors (as described in Engels et al., 2003), or retroviral SIN vectors (as described in Yu et al., 1986);
   and
lentiviral vectors (as described in Josef et al, 2008) or lentiviral SIN vectors, as described in Miyoshi et al., 1998.

Viral vectors containing TCR-encoding genes can infect lymphocytes and express functional TCR in infected lymphocytes. Preferred is a SIN vector construct.

Another example for a suitable expression construct is the Sleeping Beauty (SB) transposon transposase DNA plasmid system, SB DNA plasmid (as described in Maiti et al., 2013).

The nucleic acids and/or in particular expression constructs of the invention can also be transferred into cells by transient RNA transfection.

The nucleic acids and/or in particular expression constructs of the invention are capable of directing the synthesis/expression of the TCRs of the invention in a suitable host cell.

As discussed above, the present invention provides a cell comprising the T-cell receptor according to the present invention or the soluble T-cell receptor construct according to the present invention or the chimeric T-cell receptor of the present invention or the bi-specific antibody of the present invention or the nucleic acid(s) of the present invention or the expression construct of the present invention.

Preferably, the cell expresses the T cell-receptor of the present invention.

Preferably, the cell expresses the soluble T cell-receptor construct of the present invention.

Preferably, the cell expresses the chimeric T-cell receptor according to the present invention.

Preferably, the cell expresses the bi-specific antibody according to the present invention.

Preferably, the cell is selected from lymphocytes including but not limited to cytotoxic lymphocytes (CTLs), CD8+ T cells, CD4+ T cells, natural killer (NK) cells, natural killer T (NKT) cells, gamma/delta-T-cells.

Preferably the cell for expression of soluble T-cell receptors or bi-specific antibodies is selected from E. coli, insect cells or mammalian calls such as HEK293, which express high amounts of recombinant protein.

The transfer of GPC3-specific TCR genes into (primary) T cells by e.g. (retro-)viral vectors, SB DNA or by transient RNA transfection represents a promising means and method to generate GPC3 antigen-specific T cells. Adoptive transfer of such engineered T cells into hosts represents a promising approach for the immunotherapy of liver cancer, in particular HCC, or other cancers expressing GPC3.

The application of GPC3-specific bi-specific antibodies or soluble T-cell receptors represents an alternative promising means and method to target GPC3-positive tumor cells by redirected T cells or by the soluble T-cell receptor construct. This represents a promising approach for the immunotherapy of liver cancer, in particular HCC, or other cancers expressing GPC3.

### Pharmaceutical Compositions

As discussed above, the present invention provides a pharmaceutical composition comprising one or more of:

(i) the T-cell receptor of the present invention;
(ii) the soluble T-cell receptor construct of the present invention;
(iii) the chimeric T-cell receptor of the present invention;
(iv) the bi-specific antibody of the present invention;
(v) the nucleic acid(s) of any one of claims 7 to 10 or the expression construct of the present invention; and/or
(vi) the cell the present invention,
and, optionally, pharmaceutically excipient(s).

### Medical Uses of the GPC3-Specific TCR

As discussed above, the present invention provides the use of a T-cell receptor of the present invention, a chimeric T-cell receptor of the present invention, a bi-specific antibody of the present invention, a nucleic acid of the present invention or an expression construct of the present invention for generating genetically modified lymphocytes.

Preferably the genetically modified lymphocytes are T cells, NK cells and/or NKT cells.

In one embodiment, the generated genetically modified lymphocytes, preferably T cells and/or NK cells, are used for genetic immunization, preferably for preventing and/or treating liver cancer, in particular HCC, or other cancers expressing GPC3.

The transfer of GPC3-specific TCR genes into (primary) T cells by e.g. (retro-)viral vectors or by transient RNA transfection represents a promising means and method to generate GPC3 antigen-specific T cells. Adoptive transfer of such engineered T cells into hosts represents a promising approach for the immunotherapy of liver cancer, in particular hepatocellular carcinoma (HCC), or other cancers expressing GPC3. A new therapeutic approach is the adoptive T cell therapy of HCC. The transferred T cells can recognize tumour antigen and thereby specifically eliminate tumour cells. HCC is a potential target for T cell therapy because HCC expresses like many other tumours tumour-associated antigens, such as glypican-3 (GPC3).

In one embodiment, the chimeric T-cell receptor(s) of the present invention are used to generate genetically modified lymphocytes, preferably NK cells and/or T cells, such as CD8⁺ T cells.

The transfer of chimeric receptors containing the whole or parts of the GPC3-specific TCR genes linked to the CD3zeta chain into primary NK cells by e.g. (retro-)viral vectors or by transient RNA transfection represents a promising means and method to generate GPC3 antigen-specific NK cells. Adoptive transfer of such engineered NK cells into hosts represents a promising approach for the immunotherapy of liver cancer, in particular hepatocellular carcinoma (HCC), or other cancers expressing GPC3.

As discussed above, the present invention provides the T-cell receptor of the present invention, the soluble T-cell receptor construct of the present invention, the chimeric T-cell receptor of the present invention, the bi-specific antibody of the present invention, the nucleic acid of the present invention, the expression construct of the present invention or the host cell of the present invention for use as a medicament.

As discussed above, the present invention provides the T-cell receptor of the present invention, the soluble T-cell receptor construct of the present invention, the chimeric T-cell receptor of the present invention, the bi-specific antibody of the present invention, the nucleic acid of the present invention, the expression construct of the present invention or the host cell of the present invention for use in the detection, diagnosis, prognosis, prevention and/or treatment of liver cancer, in particular hepatocellular carcinoma (HCC), or other cancers expressing GPC3.

There are others cancers or tumors expressing GPC3, such as described by Baumhoer et al., 2008, exemplified but not restricted to:
squamous cell carcinoma of the lung,
testicular nonseminomatous germ cell tumors,
liposarcoma,
cervical intraepithelial neoplasia.

Preferably, the use comprises adoptive, target-cell specific immunotherapy or genetic immunization.

The patients are preferably HLA-A2-positive.

Methods of Detecting, Preventing and/or Treating Liver Cancer, in Particular HCC or Other Cancers Expressing GPC3

As discussed above, the present invention provides methods of preventing and/or treating liver cancer, in particular hepatocellular carcinoma (HCC) or other cancers expressing GPC3.

In one embodiment, said method comprises the steps of

(a) providing lymphocytes of a patient or a blood donor;
(b) providing one or more of
(i) a T-cell receptor of the present invention or a soluble T-cell receptor construct or a chimeric T-cell receptor of the present invention or a bi-specific antibody of the present invention,
(ii) a nucleic acid of the present invention,
(iii) an expression construct of the present invention,
(iv) a cell of the present invention, and
(v) a pharmaceutical composition of the present invention;
   (c) ex vivo introduction of one or more of (i) to (v) of step (b) into the lymphocytes of step (a) and, thereby, obtaining genetically modified lymphocytes,
   (d) administering the genetically modified lymphocytes of step (c) to a subject or patient in need thereof;
   (e) optional, combining any of the steps (b) to (d) with radiation, checkpoint inhibitor or cancer chemotherapy.

In one embodiment, said method comprises the steps of

(a) providing one or more of
(i) a T-cell receptor of the present invention or a soluble T-cell receptor construct or a chimeric T-cell receptor of the present invention or a bi-specific antibody of the present invention,
(ii) a nucleic acid of the present invention,
(iii) an expression construct of the present invention,
(iv) a cell of the present invention, and
(v) a pharmaceutical composition of the present invention;
   (b) direct application, preferably via injection or infusion, of one or more of (i) to (v) of step (a) to a subject or patient in need thereof,
   (c) optional, combining step (b) with radiation, checkpoint inhibitor or cancer chemotherapy.

In this embodiment, preferably the bi-specific antibodies or the soluble TCR constructs of the present invention are utilized. Preferably, the bi-specific antibodies or the soluble TCR constructs of the present invention are used as recombinant proteins.

In this embodiment, in step (b) the one or more of (i) to (v) of step (a) are used as recombinant proteins.

The recombinant proteins, preferably the bi-specific antibodies or the soluble TCR constructs of the present invention, are expressed, preferably in E. coli or mammalian cells, and then purified before the application.

T cells express inhibitory receptors such as cytotoxic T-lymphocyte-associated antigen 4 (CTLA-4) or programmed cell death 1 (PD-1), especially after long term exposure to the antigen. The ligands of CTLA-4 and PD-1, B7 and PD-L1 respectively, as well as the ligand of TIM-3, Galectin 9, are frequently expressed on tumor tissue and are involved in induction of T cell tolerance and thus are called immune checkpoints. To avoid silencing of transferred GPC3 specific T cells checkpoint inhibitors or immune checkpoint inhibitors, exemplified by but not restricted to anti-CTLA-4, anti-PD-L1 or anti-PD-1 antibodies, can be used to avoid inhibition of T cells within the tumor microenvironment (described in Hamid et al. (2013)).

The subjects or patients are preferably HLA-A2-positive.

Preferably, the lymphocytes provided in step (a) are T cells, NK cells and/or NKT cells, preferably CD8⁺ T cells.

The lymphocytes provided in step (a) can be obtained from the subject or patient, such as from the blood or by lymphapheresis of allogeneic blood donors.

Preferably, the ex vivo introduction in step (c) is carried out via electroporation of a nucleic acid of the present invention or of an expression construct of the present invention, Or by transfection reagents, such as liposomes, or by transient RNA transfection.

The expression construct is preferably, as described above,
a viral construct,
   preferably a lentiviral vector, retroviral vector, or other state of the art viral vector.

Examples for suitable expression constructs are viral vectors, such as
retroviral vectors (as described in Vena et al, 2004; Porter et al, 2011), such as MP71 vectors (as described in Engels et al., 2003), or retroviral SIN vectors (as described in Yu et al., 1986);
   and
lentiviral vectors (as described in Josef et al, 2008) or lentiviral SIN vectors, as described in Miyoshi et al., 1998.

Another example for a suitable expression construct is the Sleeping Beauty (SB) transposon transposase DNA plasmid system, SB DNA plasmid (as described in Maiti et al., 2013).

As discussed above, the present invention provides a method of detecting, diagnosing, prognosing, preventing and/or treating liver cancer, in particular hepatocellular carcinoma (HCC), or other cancers expressing GPC3.

Said method comprises the detection and/or destruction of liver cancer cells, in particular hepatocellular carcinoma (HCC) cells or of cancer cells of other cancers expressing GPC3, of a patient with the use of the soluble T-cell receptor construct of the present invention,

Preferably, the soluble T-cell receptor constructs are applied to patients by intravenous, subcutaneous or intramuscular infusion or injections, respectively, or by application to the mucosa of the respiratory tract by inhalation or by spraying.

Soluble T-cell receptor constructs can be applied to patients;
by intravenous infusions,
by subcutaneous injection or infusion,
by intramuscular injection,
by inhalation or by spraying to the mucosal surfaces of the respiratory tract.

Suitable dose ranges for application of soluble T-cell receptor constructs vary between 1 mg to 10 000 mg dependent on the application method and application site. Soluble T-cell receptors can bind to cells expressing GPC3 peptides on their HLA-A2 molecules, namely liver cancer cells, in particular hepatocellular carcinoma (HCC) cells, (or cells of other cancers expressing GPC3) in which GPC3 peptide 367 or GPC3 peptide 326 is presented by MHC I. Soluble T-cell receptors can induce destruction of the cells via different methods, such as radiation by linked radionuclides or killing by toxins such as pseudomonas exotoxin. In addition, soluble T-cell receptors with linked Fc-domains can redirect and activate NK-cells and/or monocytes for the lysis of liver cancer cells, in particular hepatocellular carcinoma (HCC) cells, or cells of other cancers expressing GPC3. Similarly, soluble T-cell receptors linked to anti-CD3-antibodies can redirect and activate T-cells for lysis of liver cancer cells, in particular hepatocellular carcinoma (HCC) cells, or cells of other cancers expressing GPC3.

In one embodiment the method of the invention is for detecting liver cancer, in particular hepatocellular carcinoma (HCC), or other cancers expressing GPC3,

comprising the in vitro staining of liver cancer cells, in particular hepatocellular carcinoma (HCC) cells, or cells of other cancers expressing GPC3, by using the soluble T-cell receptor construct of the present invention which is labelled.

The label can be fluorophor(s), gold and/or radionuclide(s).

Soluble T-cell receptors linked to fluorescent molecules such as fluorescein can be used to stain liver cancer cells, in particular hepatocellular carcinoma (HCC) cells, or cells of other cancers expressing GPC3 in vitro. For this purpose, liver cancer cells, in particular hepatocellular carcinoma (HCC) cells, or cells of other cancers expressing GPC3, are incubated in vitro with the soluble T-cell receptor constructs, and cells expressing HLA-A2/GPC3 peptide-complexes on the cell surface can be detected according to their fluorescence by a Fluorescence activated cell sorter (FACS) or by microscope.

Alternatively, labeled soluble T-cell receptor constructs (e.g. with flourochromes, gold or radionuclides) are injected into a subject or patient and allow detection and subsequent destruction of GP3 positive tumor, tumor metastasis or minimal residual disease after tumor resection.

The soluble TCR-constructs of the invention are suitable to be directly used for the detection and destruction of liver cancer cells, in particular hepatocellular carcinoma (HCC) cells, or cells of other cancers expressing GPC3, without the need of T-cells.

The subjects or patients are preferably HLA-A2-positive.

GPC3-Epitopes and Peptides and Vaccines and their Uses

As discussed above, the present invention provides peptides comprising at least one GPC3 epitope.

The inventors have identified immunodominant GPC3 epitopes, in particular the HLA-A2 bound GPC3 peptide 367.

The at least one GPC3 epitope comprises or consists of the amino acid sequence of SEQ ID NO. 14 (GPC3 peptide 367).

SEQ ID NO. 14 shows the amino acid sequence of the GPC3 peptide 367:

FIDKKVLKV

Glypican-3 is a protein that in humans is encoded by the GPC3 gene. The protein encoded by this gene is a member of the glypican family.

GenBank Accession No.: NM_004484.3, 580 amino acids

### UniProt Accession No.: P51654

MAGTVRTACLVVAMLLSLDFPGQAQPPPPPPDATCHQVRSFFQRLQPGL
KWVPETPVPGSDLQVCLPKGPTCCSRKMEEKYQLTARLNMEQLLQSASM
ELKFLIIQNAAVFQEAFEIVVRHAKNYTNAMFKNNYPSLTPQAFEFVGE
FFTDVSLYILGSDINVDDMVNELFDSLFPVIYTQLMNPGLPDSALDINE
CLRGARRDLKVFGNFPKLIMTQVSKSLQVTRIFLQALNLGIEVINTTDH
LKESKDCGRMLTRMWYCSYCQGLMMVKPCGGYCNVVMQGCMAGVVEIDK
YWREYILSLEELVNGMYRIYDMENVLLGLFSTIHDSIQYVQKNAGKLTT
TIGKLCAHSQQRQYRSAYYPEDLFIDKKVLKVAHVEHEETLSSRRRELI
QKLKSFISFYSALPGYICSHSPVAENDTLCWNGQELVERYSQKAARNGM
KNQFNLHELKMKGPEPVVSQIIDKLKHINQLLRTMSMPKGRVLDKNLDE
EGFESGDCGDDEDECIGGSGDGMIKVKNQLRFLAELAYDLDVDDAPGNS
QQATPKDNEISTFHNLGNVHSPLKLLTSMAISVVCFFFLVH

The amino acid sequence of GPC3 is also shown in SEQ ID NO. 15.

ATGGCCGGGACCGTGCGCACCGCGTGCTTGGTGGTGGCGATGCTGCTCA
GCTTGGACTTCCCGGGACAGGCGCAGCCCCCGCCGCCGCCGCCGGACGC
CACCTGTCACCAAGTCCGCTCCTTCTTCCAGAGACTGCAGCCCGGACTC
AAGTGGGTGCCAGAAACTCCCGTGCCAGGATCAGATTTGCAAGTATGTC
TCCCTAAGGGCCCAACATGCTGCTCAAGAAAGATGGAAGAAAAATACCA
ACTAACAGCACGATTGAACATGGAACAGCTGCTTCAGTCTGCAAGTATG
GAGCTCAAGTTCTTAATTATTCAGAATGCTGCGGTTTTCCAAGAGGCCT
TTGAAATTGTTGTTCGCCATGCCAAGAACTACACCAATGCCATGTTCAA
GAACAACTACCCAAGCCTGACTCCACAAGCTTTTGAGTTTGTGGGTGAA
TTTTTCACAGATGTGTCTCTCTACATCTTGGGTTCTGACATCAATGTAG
ATGACATGGTCAATGAATTGTTTGACAGCCTGTTTCCAGTCATCTATAC
CCAGCTAATGAACCCAGGCCTGCCTGATTCAGCCTTGGACATCAATGAG
TGCCTCCGAGGAGCAAGACGTGACCTGAAAGTATTTGGGAATTTCCCCA
AGCTTATTATGACCCAGGTTTCCAAGTCACTGCAAGTCACTAGGATCTT
CCTTCAGGCTCTGAATCTTGGAATTGAAGTGATCAACACAACTGATCAC
CTGAAGTTCAGTAAGGACTGTGGCCGAATGCTCACCAGAATGTGGTACT
GCTCTTACTGCCAGGGACTGATGATGGTTAAACCCTGTGGCGGTTACTG
CAATGTGGTCATGCAAGGCTGTATGGCAGGTGTGGTGGAGATTGACAAG
TACTGGAGAGAATACATTCTGTCCCTTGAAGAACTTGTGAATGGCATGT
ACAGAATCTATGACATGGAGAACGTACTGCTTGGTCTCTTTTCAACAAT
CCATGATTCTATCCAGTATGTCCAGAAGAATGCAGGAAAGCTGACCACC
ACTATTGGCAAGTTATGTGCCCATTCTCAACAACGCCAATATAGATCTG
CTTATTATCCTGAAGATCTCTTTATTGACAAGAAAGTATTAAAAGTTGC
TCATGTAGAACATGAAGAAACCTTATCCAGCCGAAGAAGGGAACTAATT
CAGAAGTTGAAGTCTTTCATCAGCTTCTATAGTGCTTTGCCTGGCTACA
TCTGCAGCCATAGCCCTGTGGCGGAAAACGACACCCTTTGCTGGAATGG
ACAAGAACTCGTGGAGAGATACAGCCAAAAGGCAGCAAGGAATGGAATG
AAAAACCAGTTCAATCTCCATGAGCTGAAAATGAAGGGCCCTGAGCCAG
TGGTCAGTCAAATTATTGACAAACTGAAGCACATTAACCAGCTCCTGAG
AACCATGTCTATGCCCAAAGGTAGAGTTCTGGATAAAAACCTGGATGAG
GAAGGGTTTGAAAGTGGAGACTGCGGTGATGATGAAGATGAGTGCATTG
GAGGCTCTGGTGATGGAATGATAAAAGTGAAGAATCAGCTCCGCTTCCT
TGCAGAACTGGCCTATGATCTGGATGTGGATGATGCGCCTGGAAACAGT
CAGCAGGCAACTCCGAAGGACAACGAGATAAGCACCTTTCACAACCTCG
GGAACGTTCATTCCCCGCTGAAGCTTCTCACCAGCATGGCCATCTCGGT
GGTGTGCTTCTTCTTCCTGGTGCACTGA

The nucleotide sequence of GPC3 is also shown in SEQ ID NO. 16.

In one embodiment, the amino acid sequence of the epitope has a length of at least 8 amino acids. In one embodiment, the amino acid sequence of the epitope has a length of 9 amino acids. Preferably, the amino acid sequence of the epitope has a length of 8, 9 or 10 amino acids.

In an embodiment, the peptide has a length in the range of 5 to 50 amino acids, preferably 8 to 25 amino acids, more preferably 8 to 15 amino acids or 9 to 15 amino acids. In an embodiment, the peptide is a nonamere, i.e. has a length of 9 amino acids.

In one embodiment, for MHC I presentation (which is a preferred embodiment), the peptide has a length of 8 to 10 amino acids, preferably 9 amino acids.

In one embodiment, for MHC II presentation, the peptide has a length of 15 to 24 amino acids, preferably 18, 19 or 20 amino acids.

In one embodiment, the peptide of the present invention comprises further GPC3 epitope(s), such as a GPC3 epitope comprising or consisting of the amino acid sequence of SEQ ID NO. 17 (GPC3 peptide 326).

For example, the peptide of the present invention can comprise or consist of the amino acid sequence of SEQ ID NO. 14 (GPC3 peptide 367) and the amino acid sequence of SEQ ID NO. 17 (GPC3 peptide 326).

SEQ ID NO. 17 shows the amino acid sequence of the GPC3 peptide 326:

TIHDSIQYV

In one embodiment, the peptide comprises further component(s), such as
tag(s),
   such as 6-His-tag, Flag-tag,
linker,
   such as polylinkers to link one peptide/epitope to another peptide/epitope
label(s),
   such as radioisotopes, enzymes, gold, luminescent or fluorescent labels,
N- and/or C-terminal modification(s),
   such as comprising acetylation and/or amidation of the N- and/or C-terminus,
drug(s),
   such as tumor chemotherapeutics, kinase inhibitors,
agent(s),
and/or
combinations thereof.

The skilled artisan will be able to select suitable further components.

Said further component(s) can be covalently attached to the peptide of the vaccine.

In one embodiment, the peptide comprises modified amino acid(s), unnatural amino acid(s) or peptidomimetic(s).

As discussed above, the present invention provides a nucleic acid molecule coding for at least one peptide according to the present invention or a plasmid comprising at least one such nucleic acid molecule.

As discussed above, the present invention provides an antibody against a peptide according to the present invention.

As discussed above, the present invention provides a composition comprising

(i) at least one peptide according to the present invention or at least one nucleic acid according to the present invention,
(ii) optionally, a carrier,
(iii) optionally, an adjuvant.

Preferably, the composition comprises at least two peptides according to the present invention or at least two nucleic acids according to the present invention.

The compositions according to the invention can further comprise
a carrier
and/or
an adjuvant.

Suitable adjuvants are known in the art.

Since peptides are usually poor immunogens, the efficacy of peptide-based vaccine depends on the adequate presentation of the epitopes to the immune system.

The skilled artisan will be able to select suitable carriers) and/or adjuvant(s).

In one embodiment, the carrier is a virus particle or parts thereof, an envelope protein of a viral vector or of a virus particle, a nanocarrier or a liposomal compound enveloping the peptide(s) in a micellular structure.

As discussed above, the present invention provides the peptide of the present invention, the nucleic acid molecule of the present invention or the pharmaceutical composition of the present invention for use in medicine.

As discussed above, the present invention provides the peptide of the present invention, the nucleic acid molecule of the present invention or the pharmaceutical composition of the present invention for use as a vaccine.

As discussed above, the present invention provides a vaccine comprising at least one peptide of the invention, a nucleic acid molecule of the present invention or the pharmaceutical composition of the present invention.

The vaccines are preferably peptide-based vaccines.

The vaccines of the present invention can further comprise excipient(s), such as pharmaceutically acceptable excipient(s), which can be necessary for the different administration regimes (e.g. via injection, such as subcutaneously or intradermally).

Further suitable excipient(s) are known to the skilled artisan.

A peptide of the invention or the peptide comprised in a vaccine of the invention does not comprise the full-length glypican-3 (GPC3) protein.

As discussed above, the present invention provides the use of the vaccine of the present invention for preventing and/or treating liver cancer, in particular hepatocellular carcinoma (HCC) or other cancers, expressing GPC3.

As discussed above, the present invention provides a method of preventing and/or treating liver cancer, in particular hepatocellular carcinoma (HCC), or other cancers expressing GPC3,
comprising the step of
   administering a peptide of the present invention, a nucleic acid molecule of the present invention, a pharmaceutical composition of the present invention or a vaccine of the present invention
to a subject in need thereof.

### Further Description of Preferred Embodiments

The inventors have identified T cell clones that specifically recognise Glypican-3 expressing cells and demonstrate effector functions such as cytokine production and effective killing of target cells. The T cell clones were generated using an allorestricted stimulation of T cells with peptide or snRNA pulsed dendritic cells (DCs).

After successful generation and characterisation of functional T cell clones, the T cell receptor (TCR) sequence was sequenced and subsequently cloned into a retroviral-vector construct. The retroviral-vector based transfer of previously cloned T cell receptors can be used to equip T cells with the GPC3-specific effector function. Functionality of transduced T cells was evaluated in vitro.

The identified GPC3 specific TCRs are suitable for adoptive T cell therapy of HCC. HCC expresses like many other tumours tumour-associated antigens (TAAs), one of them is glypican-3 (GPC3). T cells that would naturally respond to TAAs are removed from the system in a process called negative selection during T cell development because TAAs are derived from self-tissue. Nevertheless T cells can be reactivated against tumours by genetic engineering more precisely insertion of a TAA-specific T cell receptor.

T cells carrying the TCRs of the present invention can recognize and specifically eliminate GPC3 expressing tumour cells. Genes coding for the GPC3 specific TCRs of the invention can be inserted into patients' T cells to enable them to recognize and kill cancer cells, especially HCC. Genetic modifications of the TCRs of the present invention to improve migration of T cells into the tumour tissue, to enhance proliferation or resistance to tumour-derived immunosuppressive factors are optional. Additionally, the insertion of suicidal genes that allow the elimination of transferred T cells may be advantageous. T cells that were ex vivo equipped with the TCRs of the present invention can be transferred back to the patient where they can exert their anti-tumour activity.

According to the present invention, GPC3 peptide 367 presented by MHC-I is recognized by T cells carrying the T cell receptors of the present invention or GPC3 peptide 326 presented by MHC-I is recognized by T cells carrying the T cell receptors of the present invention, respectively. Furthermore it has been shown that GPC3+ human hepatoma cells are effectively killed by said T cells. Hence, adoptive T-cell immunotherapy with the TCRs of the present invention allows recognition as well as cytotoxic elimination of glypican-3 (GPC3) expressing malignant cells, which makes the TCRs of the present invention a very promising tool to treat HCC patients.

### Identification of Immunodominant GPC3 Peptides

Immunodominant epitopes for GPC3 have not been described yet. In this study we used Ultra Nano HPLC coupled on-line to the Q Exactive mass spectrometer to obtain a comprehensive HLA class I peptidome from a GPC3 and HLA-A2 positive hepatoma cell line. The resulting data were analysed using the MaxQuant bioinfonnatics platform.

Two HLA-A2 bound GPC3 peptides were identified, referred to as “GPC3 peptide 367” (GPC3₃₆₇: FIDKKVLKV) and “GPC3 peptide 326” (GPC3₃₂₆: TIHDSIQYV), wherein GPC3 peptide 326 is described in Komori et al., 2006.

The knowledge of these two peptides enables us to target GPC3 epitopes that are presented on GPC3 positive HCC cells. See also FIG. 2.

### T Cells and TCR Specific for Peptide GPC3₃₆₇

We have identified and cloned the T cell receptor (TCR) specific for GPC3 peptide 367 from respective T cell clones. This TCR is also referred to as P1-1. The T cell receptor sequence coding for the variable parts of alpha and beta chain of P1A was codon optimized to increase the level of expression after viral transfer into T cells. Furthermore the constant domains were exchanged with murine constant domains to avoid miss pairing with endogenous TCR chains and hence ensure optimal expression (see FIG. 5).

T cells engrafted with this optimized GPC3-specific TCR P1-1 showed strong GPC3 MHC streptamer binding. When co-cultured with GPC3 peptide loaded target cells or a GPC3 expressing hepatoma cell line (HepG2), TCR P1-1 transduced T cells secreted IFNγ, TNFα and IL2 and delocalized the degranulation marker Lamp-1 (see FIG. 6A and FIG. 8).

Cytotoxicity was observed by killing of HepG2 cells that are GPC3 and HLA-A2 positive. The cytotoxic effect depends on GPC3 peptide presentation on HLA-A2 since no killing was observed on HLA-A2-GPC3+ Huh7 cells (see FIG. 6B). Killing of target cell already occurred at low effector:target (E:T) ratios of 0.1:1 and up to 60% of killing was reached when an E:T ratio of 1:1 was used. More detailed investigation of the killing capacity showed very rapid killing of around 90% of target cells when E:T ratios of 1:1 or higher were used. At lower E:T ratios around 50% of target cells were killed by P1-1 equipped T cells (FIG. 7A). To analyse the TCR avidity TCR P1-1 transduced T cells were incubated with T2 cells loaded with peptide GPC3 367 in different concentration. A clear concentration dependent IFNγ secretion was observed that indicated a medium avidity of the TCR of the present invention (FIG. 7B).

### TCR Specific for Peptide GPC3₃₂₆

We have identified and cloned the T cell receptor (TCR) specific for GPC3 peptide 326 from respective T cell clones. This TCR is also referred to as P2-1.

The following examples and drawings illustrate the present invention without, however, limiting the same thereto.

### BRIEF DESCRIPTION OF THE DRAWINGS

FIG. 1

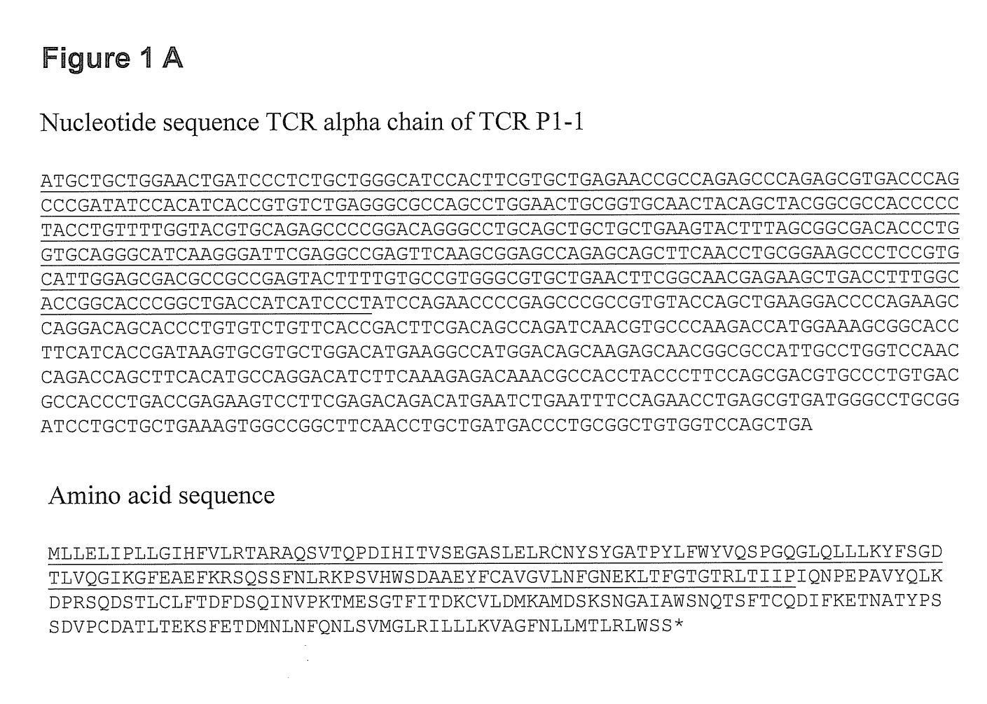

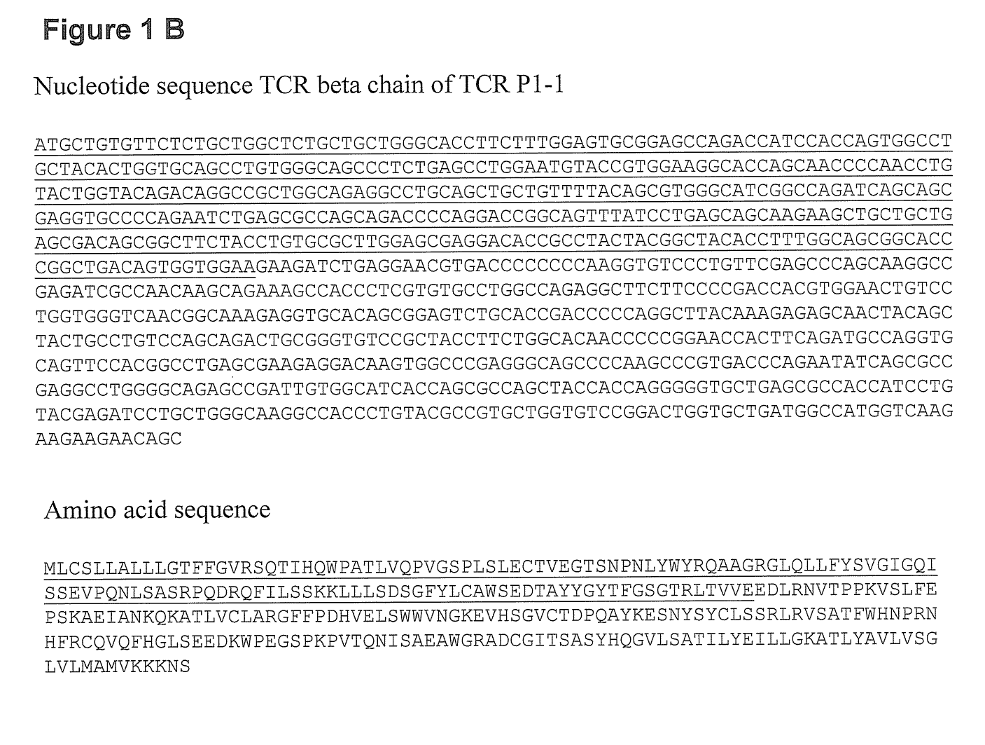

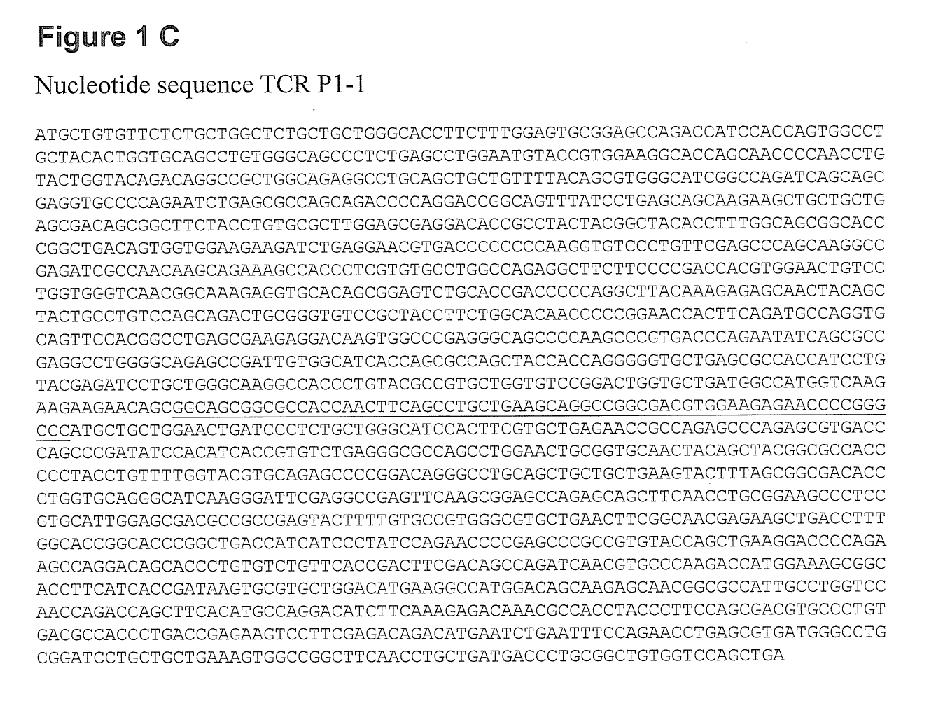

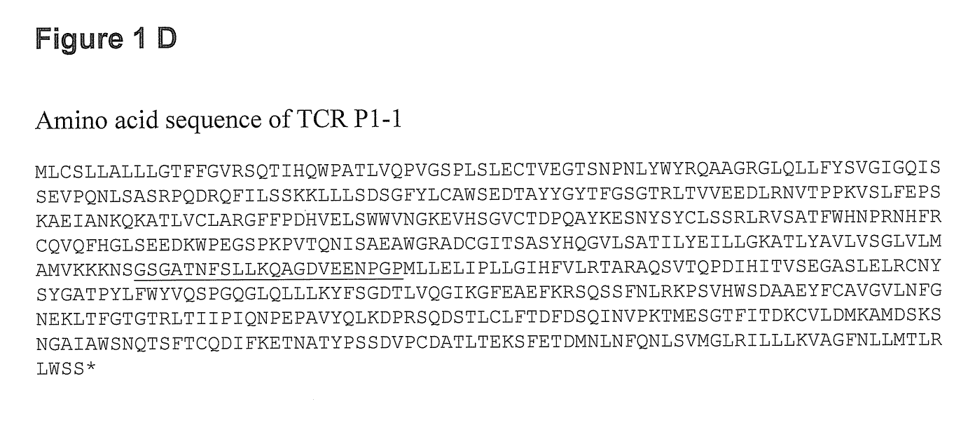

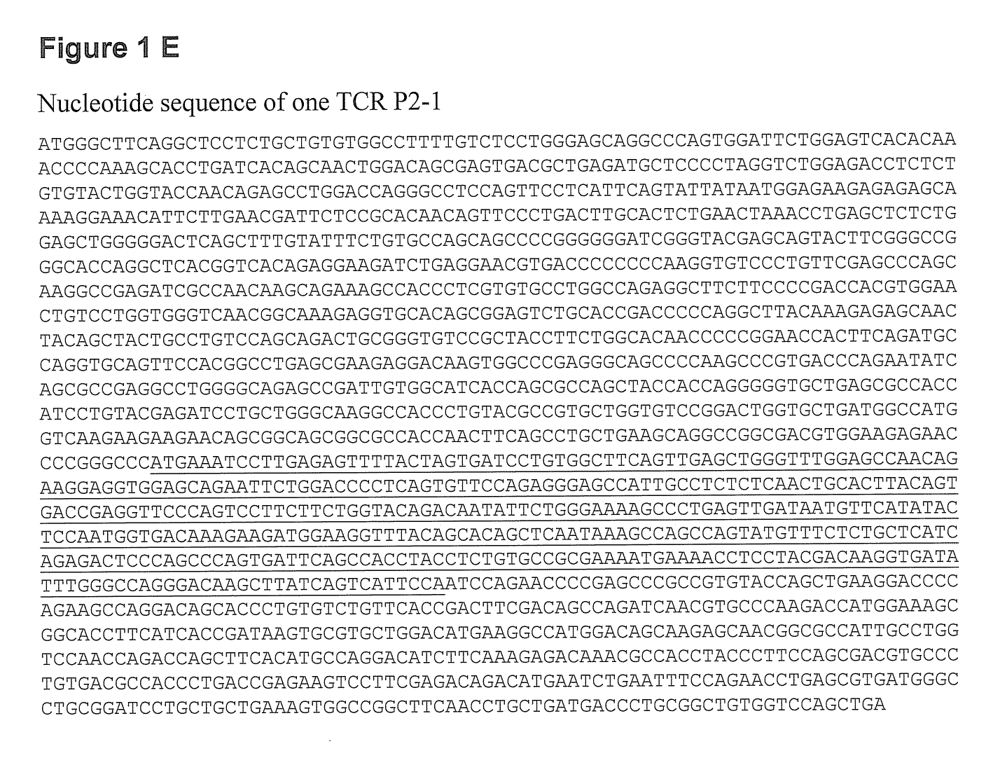

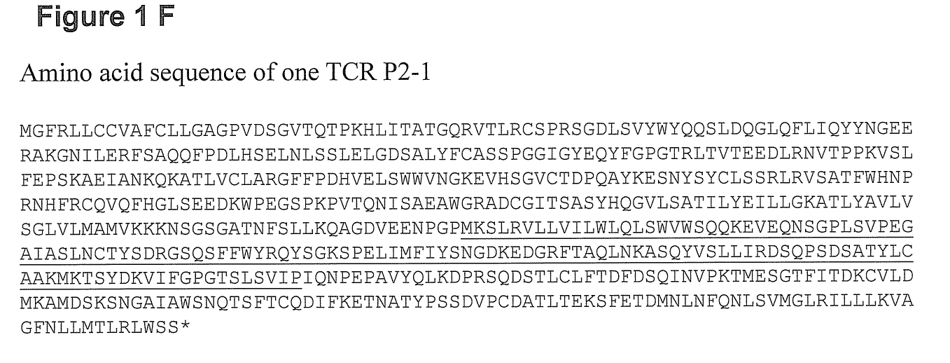

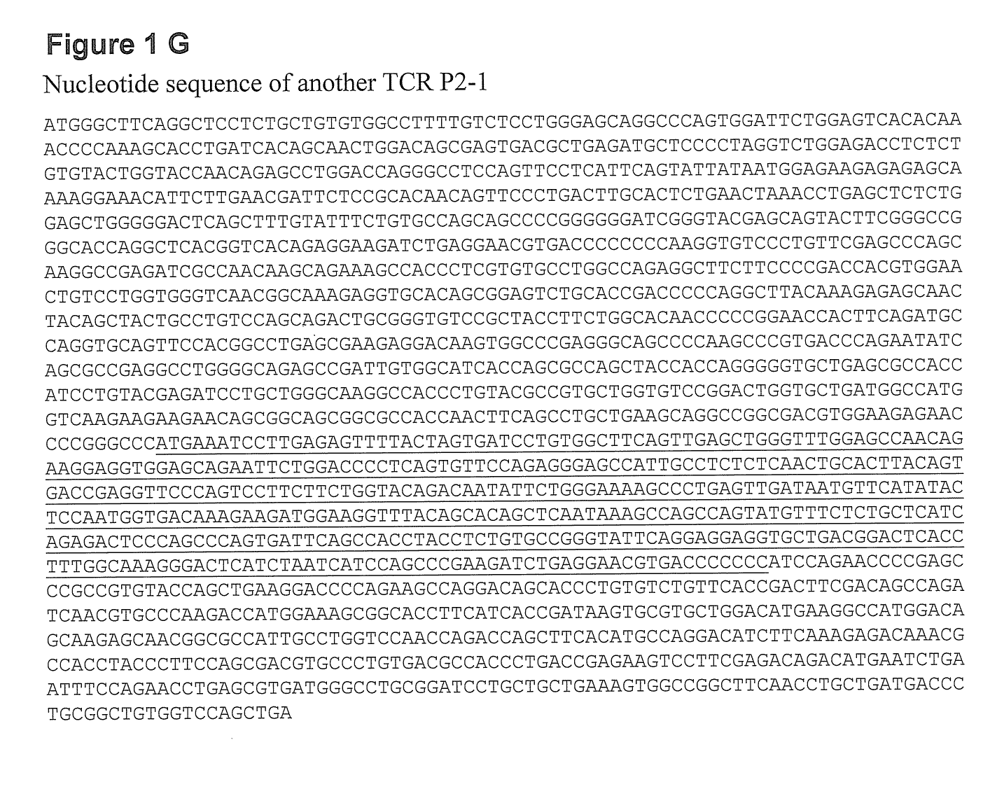

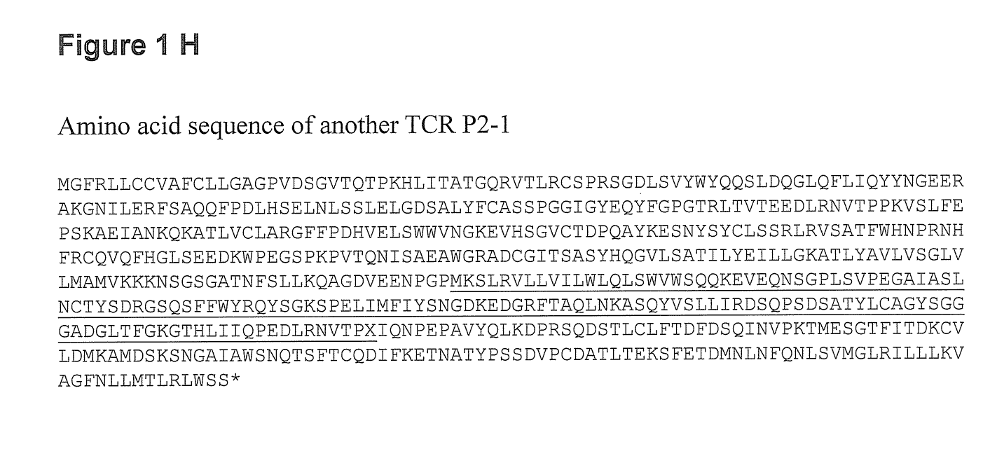

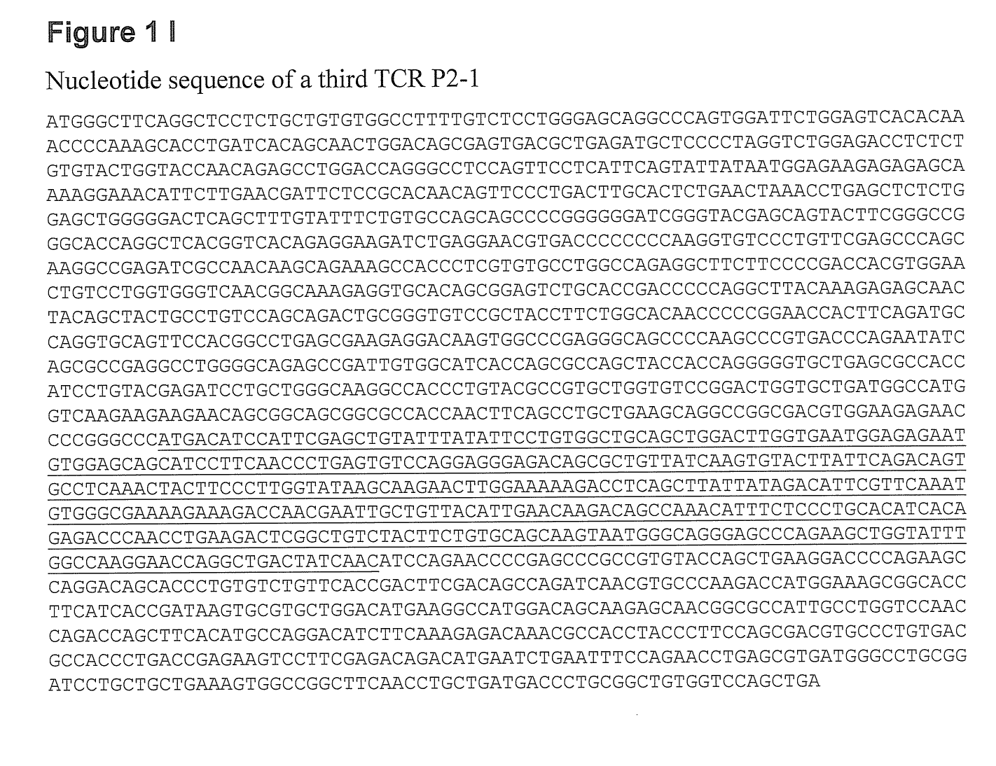

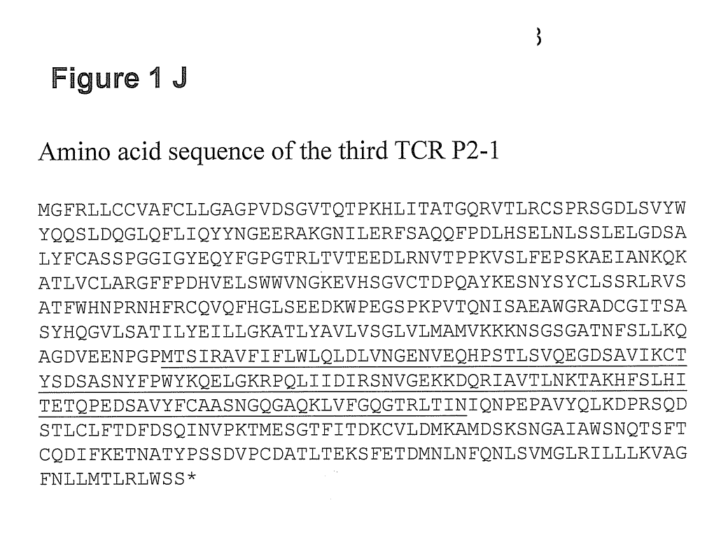

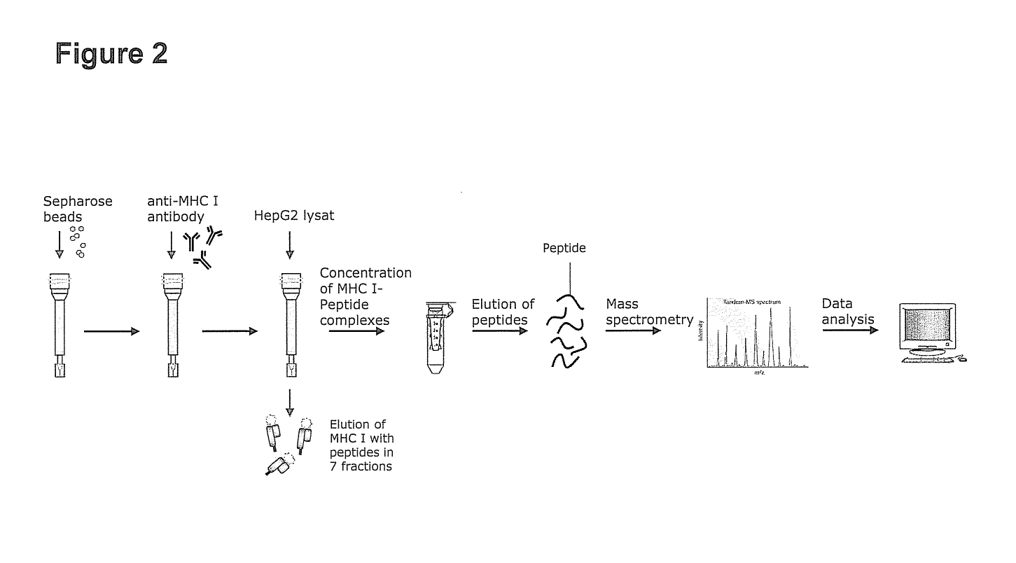

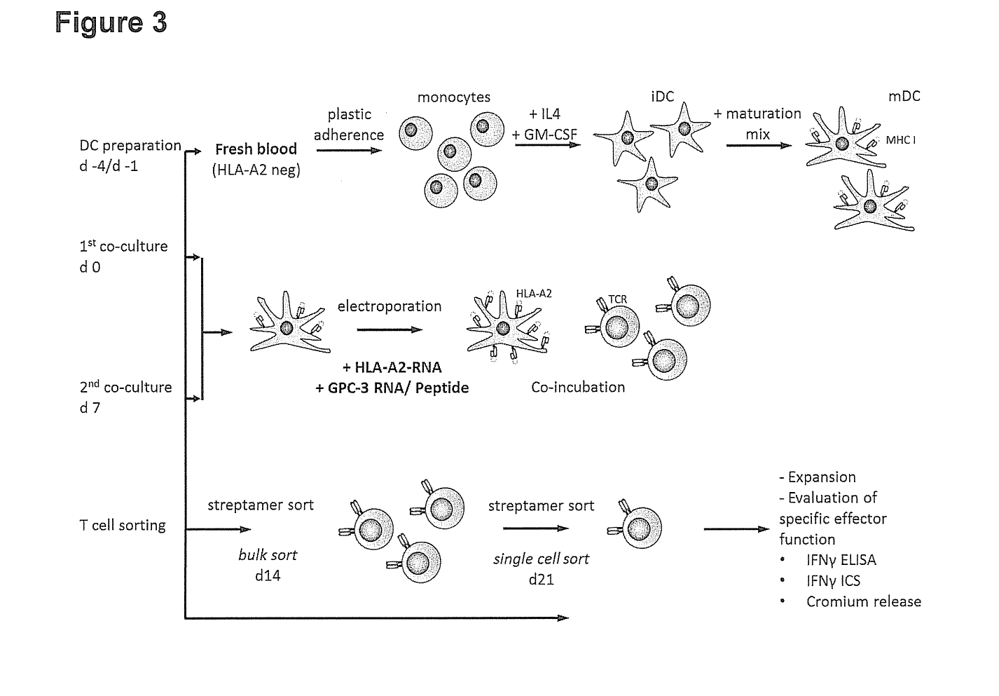

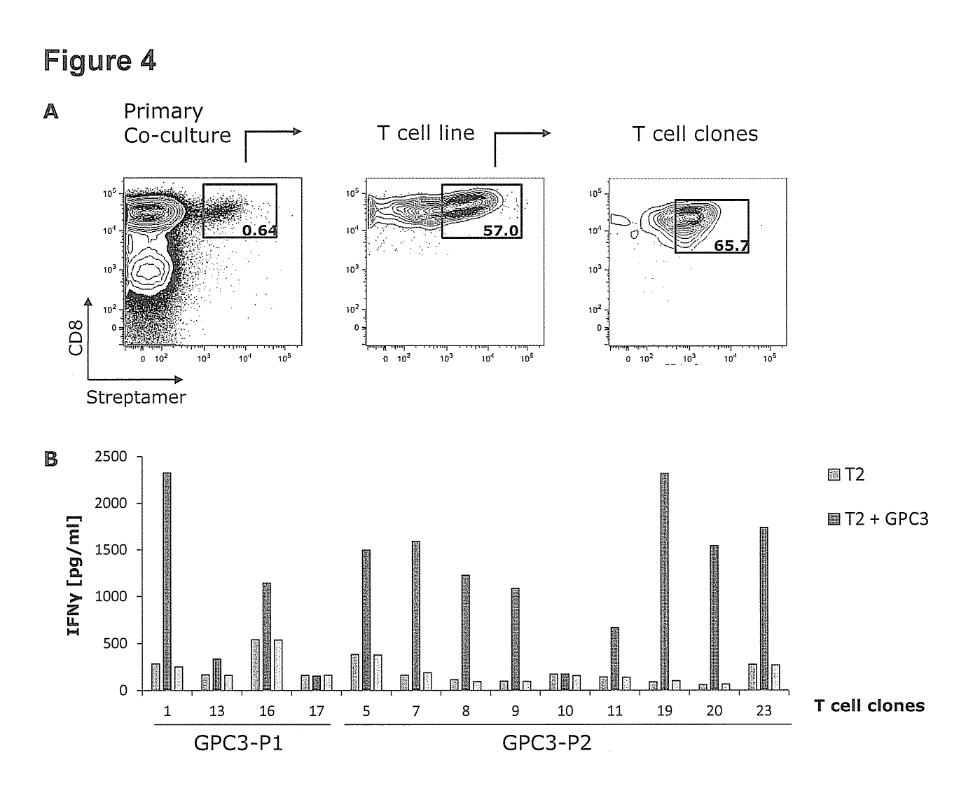

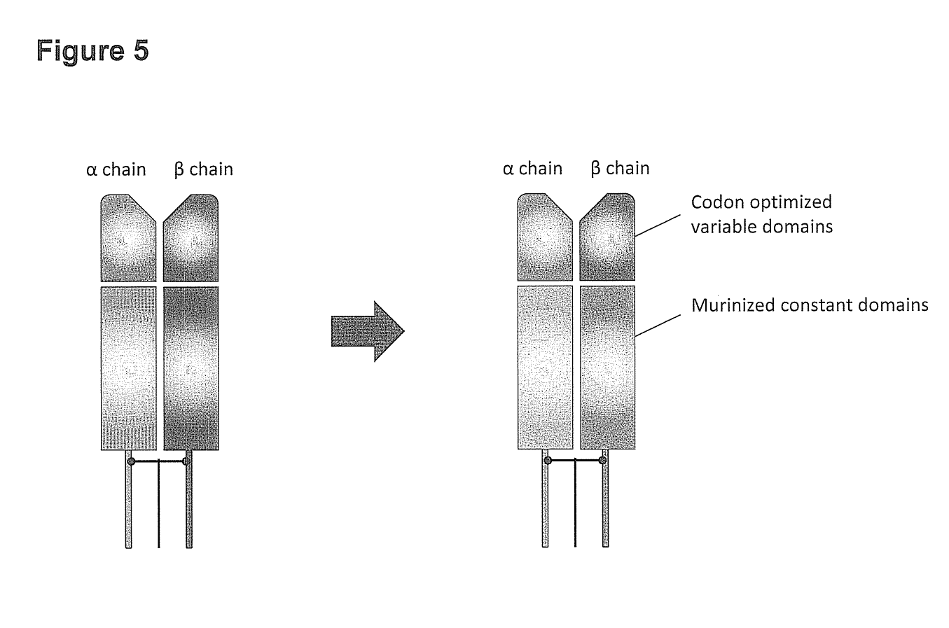

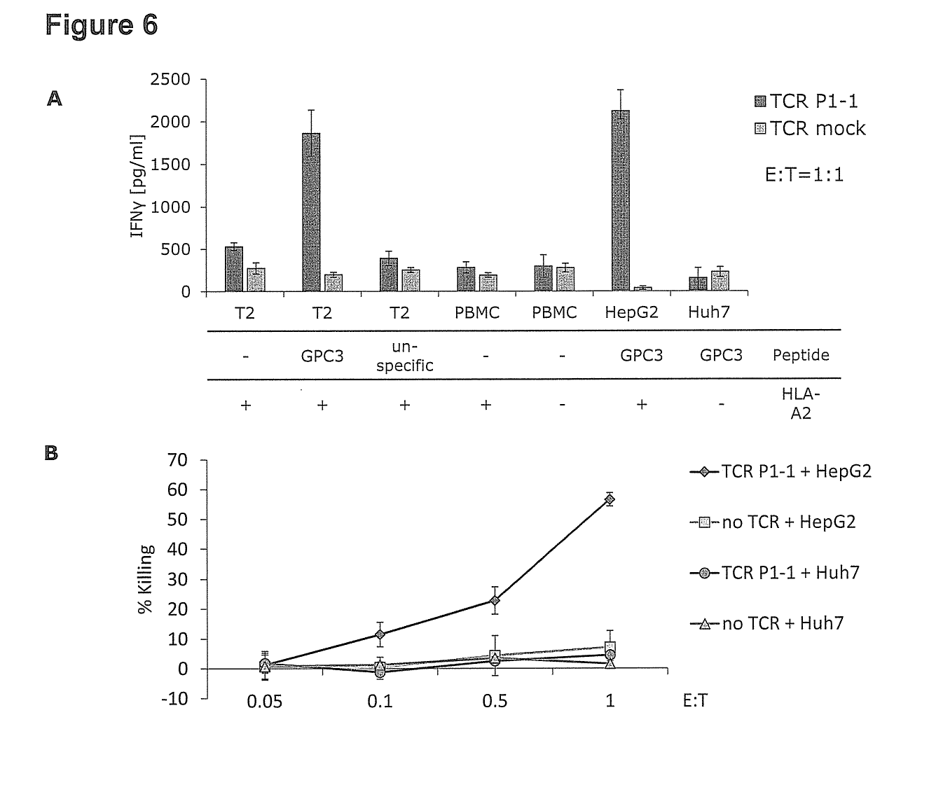

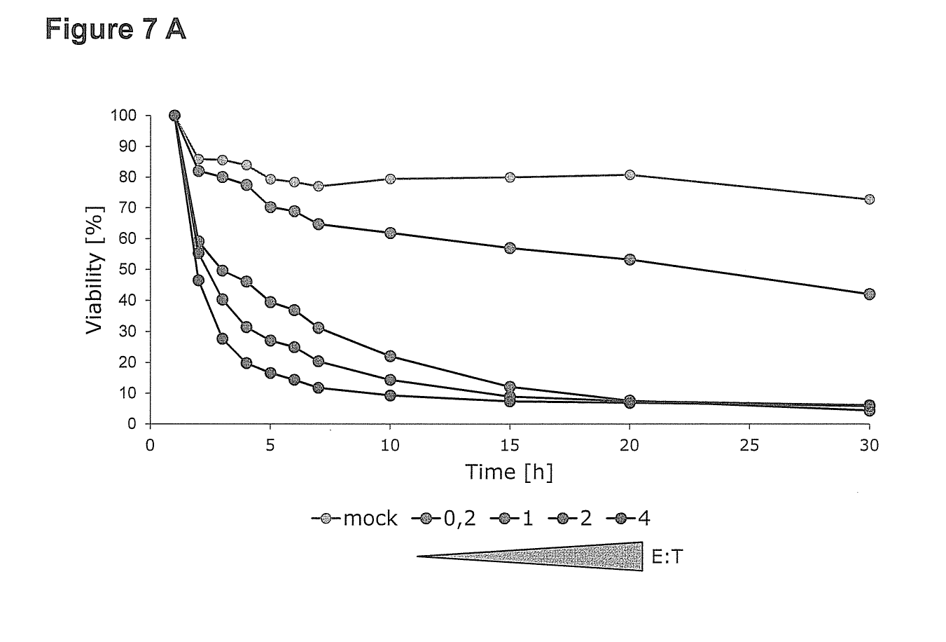

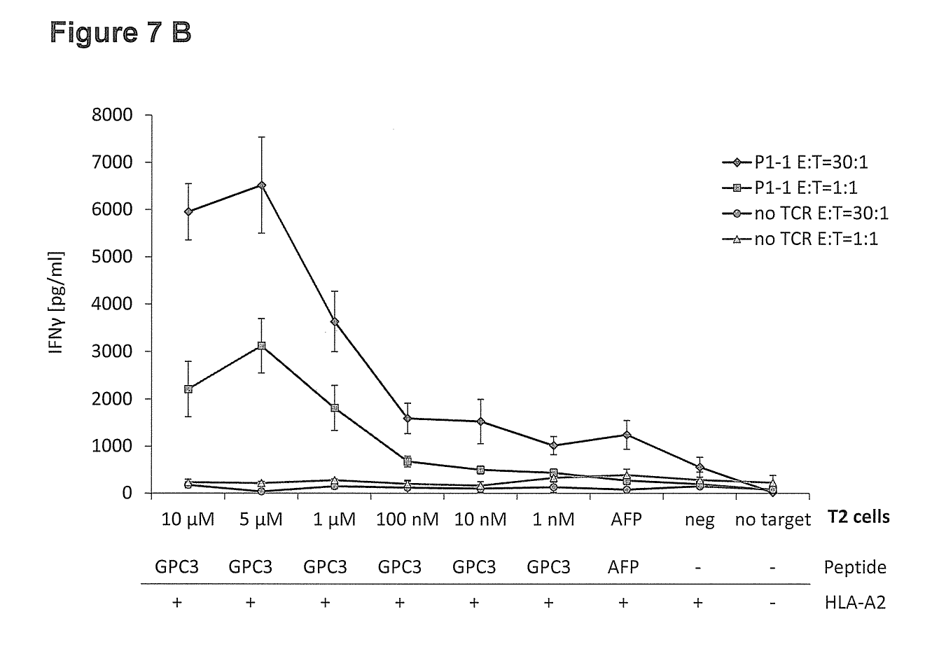

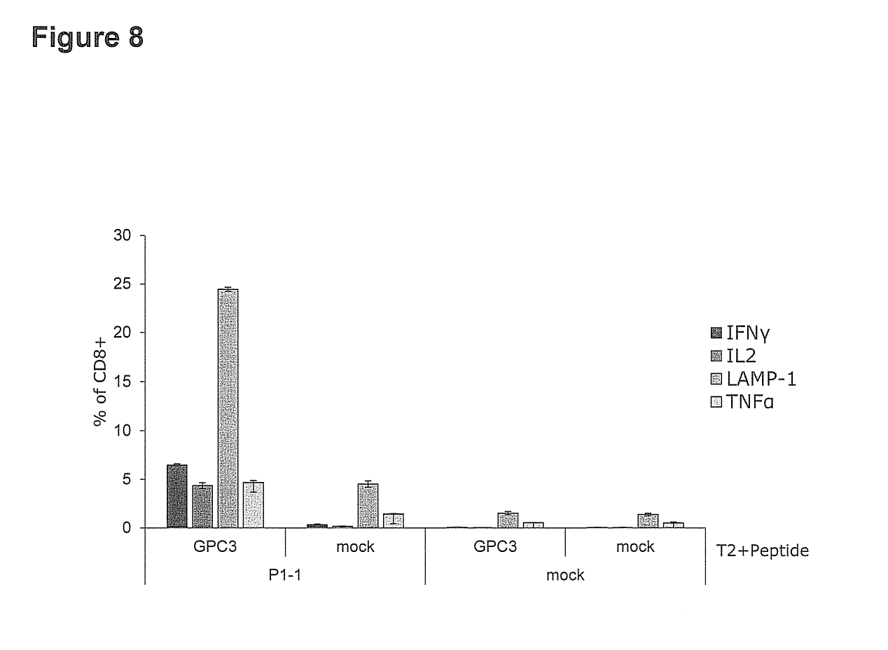

A to D) Sequence of the TCR P1-1

A) Sequence of the TCR P1-1 alpha chain.

Nucleotide and amino acid sequence of the TCR alpha chain: underlined is the variable alpha region.

B) Sequence of the TCR P1-1 beta chain.

Nucleotide and amino acid sequence of the TCR beta chain: underlined is the variable beta region.

C and D) Sequence of the TCR P1-1.

Nucleotide and amino acid sequence of the TCR: underlined is the linker region.

E to J) Sequences of the TCR P2-1

E and F) Sequence of one TCR P2-1.

Nucleotide and amino acid sequence of the TCR: underlined is the TCR alpha chain.

G and H) Sequence of another TCR P2-1.

Nucleotide and amino acid sequence of the TCR: underlined is the TCR alpha chain.

I and J) Sequence of a third TCR P2-1.

Nucleotide and amino acid sequence of the TCR: underlined is the TCR alpha chain.

FIG. 2. Schematic illustration of mass spectrometric analysis of MHC I bound peptides on HepG2 cells.

MHC-I peptide complexes were isolated from total cell lysate by sepharose bound anti-MHC I antibodies. After concentration, peptides were eluted from MHC I and subsequently analysed using Ultra Nano HPLC coupled on-line to the Q Exactive mass spectrometer.

FIG. 3. Time schedule for the allorestricted stimulation of T cells.

according to Wilde et al., 2012. DCs are prepared from fresh blood, maturated and electroporated with HLA-A2 RNA. GPC3 can be loaded either in form of peptide or GPC3 mRNA can be electroporated together with HLA-A2. Ten hours after electroporation DCs are co-incubated with CLTs from the same HLA-A2 negative donor. On day 14 and 21, the GPC3-specific HLA-A2-restricted T cell cultures are stained with multimer and sorted via fluorescence activated cell sorting. The sorted T cells are cloned in limiting dilution cultures or expanded as bulk lines. Functional assay for evaluation of their specific effector functions are performed.

FIG. 4.

A) Streptamer staining (P1; 367) of stimulated T cells, corresponding T cell lines, and one exemplary T cell clone. Streptamer binding T cell population was enriched from 0.6% in the primary co-culture to 57% in the T cell line. T cell clones showed strong streptamer binding

B) T cells clones specific for P1 or P2 were identified in an IFNγ secretion assay. Specific T cell clones released IFNγ on T2 cells loaded with GPC3 peptides but not on T2 cells without or with control peptides.

FIG. 5. TCR Modifications:

1) Codon optimization for higher expression levels.

2) Murinized constant domains to avoid miss pairing with endogenous TCR chains.

FIG. 6.

A) P1-1 transduced T cells specifically secrete IFNγ when co-incubated with T2 cells loaded with GPC3 peptide 367 but not on other target cells.

B) Killing of GPC3+ human hepatoma cells by T cells engrafted with the GPC3₃₆₇ specific TCR P1-1. GPC3+ HLA-A2+ HepG2 cells and GPC3+ HLA-A2− Huh7 cells were incubated with P1-1 transduced T cells at different effector target (E:T) ratios. P1-1 enables T cells to effectively kill target cells at low E:T ratios in an HLA-A2 dependent manner.

FIG. 7.

A) Killing of GPC3+ human hepatoma cells by T cells engrafted with the GPC3 367 specific TCR P1-1. GPC3+ HLA-A2+ HepG2 cells were incubated with P1-1 transduced T cells at different effector target (E:T) ratios and target cell viabitity was measured over time. P1-1 enables T cells to rapidily kill target cells at low E:T ratios.

B) P1-1 transduced T cells specifically secrete IFNγ when co-incubated with T2 cells loaded with GPC3 peptide 367 in a peptide concentration dependent fashion.

FIG. 8.

P1-1 transduced T cells specifically secrete IFNγ, IL-2 and TNF-α and delocalize the degranulation marker LAMP-1 when co-incubated with T2 cells loaded with GPC3 peptide 367 but not on mock peptide loaded target cells.

### EXAMPLES

### 1. Material and Methods

### 1.1 PBMC and Cell Lines

Peripheral blood mononuclear cells (PBMC) from healthy human donors were isolated by Ficoll density gradient centrifugation.

T2 is a HLA-A2+ TAP deficient human lymphoma cell line that was used as a target in IFNγ-release assays.

LCL-B27 is a HLA-B27+ B-lymphoblastoid cell line that was used as a feeder cell line for unspecific T cell restimulation.

Human Embryonic Kidney 293 (293T) cells were used as producer cell line of virus supernatant for T cell transduction.

HepG2 is a human hepatoma cell line expressing HLA-A2 and GPC3, which was used as a target in killing assays.

Mammalian cell culture was performed under sterile cell culture conditions and cell were incubated at 37° C. and 5% CO₂.

### 1.2 Media

All T cell cultures were maintained in human T cell medium (hTCM) compromised of RPMI1640 medium supplemented with 10% human serum, 4 mM L-glutamine, 100 IU/ml penicillin, 100 μg/ml streptomycin, 6 μg/ml gentamycin, 1 mM sodium pyruvate and 1× Non-Essential Amino Acids (NEAA). Human serum was isolated in our laboratory from healthy donors.

Dendritic cell cultures were maintained in DC medium consisting of very low endotoxin RPMI 1640 medium supplemented with 1.5% human serum, 100 IU/ml penicillin and 100 μg/ml streptomycin.

LCL and T2 cells were maintained in RPMI1640 medium supplemented with 10% fetal bovine serum, 4 mM L-glutamine, 100 IU/ml penicillin, 100 μg/ml streptomycin, 1 mM sodium pyruvate and 1×NEAA.

HepG2 cells were maintained in DMEM medium supplemented with 10% fetal bovine serum, 4 mM L-glutamine, 100 IU/ml penicillin, 100 μg/ml streptomycin, 1 mM sodium pyruvate and 1×NEAA.

### 1.3 Cytokines, Peptides Multimers and Antibodies

Recombinant human IL-4 and IL-6 were obtained from GellGenix. Recombinant human GM-CSF, IL-7, IL-15, TNFα, IL-1β, PGE₂ and IL-2 were purchased from Genzyme, Promokine, Peprotech, Miltenyi Biotec, R&D, Pfizer and Novartis, respectively.

The HLA-A2-restricted peptides representing defined epitopes from GPC3 (367: FIDKKVLKV; 326: TIHDSIQYV) were synthesized by iba.

HLA-A2-GPC3-multimers were generated by AG Busch, Institute for Medical Microbiology, Immunology and Hygiene, TU Munich (Knabel et al., 2002). Strap-Tactin PE for labelling of MHC-I multimers was purchased from iba.

Anti-human CD8-APC, CD8-PE, anti-CD14, anti-CD83 and anti-CD80 antibodies were obtained from BD, Immunotools, eBioscience and Immunotech, respectively.

Propidiumiodide (1 mg/ml) was obtained from Roth.

Anti-human CD3 (OKT3) was kindly provided by AG Kremmer, Institute for Molecular Immunology, Helmholtz Zentrum Munich.

### 1.4 Generation of Dendritic Cells (DC) for T Cell Stimulation

To generate DC PBMC from a healthy HLA-A2 negative donor were resuspended in DC medium and plated at 5×10⁶ cell per 75 cm² NUNC culture flask. After incubated for 1.5 hours at 37° C. non-adherent cells were removed by extensive washing. The remaining adherent monocytes were cultured in medium containing 100 ng/ml GM-CSF and 20 ng/ml IL-4 for 24 hours. On day 2 immature DC were differentiated by addition of maturation cytokines (10 ng/ml TNFα, 10 ng/ml IL-1β, 15 ng/ml IL-6 and 1 μg/ml PGE2) directly to the culture. DC were incubated for an additional day before mature DC were harvested. Maturation was confirmed by staining with anti-CD14, anti-CD83 and anti-CD80 and subsequent flow cytometry a FACSCanto II (BD Biosciences).

Mature DC were transfected with GPC3 and HLA-A2 by RNA electroporation. 2×10⁶ cells were resuspended in 200 μl OptiMEM I medium and mixed with 25 μg HLA-A2 RNA and 50 GPC3 RNA. The DC-RNA mixture was placed into a 0.4 cm electroporation cuvette and incubated on ice for 3 min. Electroporation was performed with the Gene Pulser Xcell (Bio-Rad) using the exponential electroporation protocol at 300 V and 150 HLA-A2 RNA and GPC3 RNA were in vitro (ivt) transcribed using the mMES SAGE mMACHINE T7 kit and Poly(A) tailing kit (both Ambion), according to the manufacturer's instructions. HLA-A2 and GPC3 expression was assed via flow cytometry.

### 1.5 Allorestricted Stimulation of T Cells

CD8+ T lymphocytes were enriched from total PBMCs via negative selection using the Dynabeads® Untouched™ Human CD8 T Cells Kit from Invitrogen. Primary cultures contained 1×10⁶ CD8+ T cells and 1×10⁵ HLA-A2 and GPC3 ivt-RNA pulsed mature DC in 2 ml of hTCM medium per well of a 24-well tissue culture plate. 5 ng/ml IL-7 was added immediately to the stimulation on day 0. 50 IU/ml IL-2 were added on day 2 and thereafter every 3^{rd} day. Primed T cells were restimulated seven days later using freshly generated ivt-RNA pulsed mature DC.

### 1.6 Multimer Staining and Cloning of T Cells

After 14 days of T cell stimulation HLA-A2-restricted GPC3-specific T cells were detected by MHC-I-multimer staining. GPC3 MHC-multimers were labelled with streptactin-PE prior to staining. For each 1×10⁶ T cells 0.2 μg PE-labelled GPC3 MHC-multimers were used. T cells were incubated with PE-labelled GPC3 MHC-multimers for 20 min in 50 μl PBS+0.5% human serum on ice and in the dark. APC-labelled anti-human CD8 was added and incubated for an additional 20 min, before washing and analysis by flow cytometry. Dead cells were excluded by propidium iodide staining. For sorting up to 10×10⁶ cells were stained as described above and sorted on a FACS Aria sorter (BD Biosciences). Sorted CD8+ GPC3 MHC-multimer+ T cells were cloned by limiting dilution. Therefore 0.3 CD8+ GPC3 MHC-multimer+ T cells were seeded per well of a 96-well round-bottom plate. In Addition each well contained a feeder mixture consisting of 1×10⁵ allogeneic PBMC (irradiated to 35 Gy), 1×10⁴ LCL-B27 (irradiated to 50 Gy), 30 ng/ml OKT3 and 50 IU/ml IL-2 in 200 n1 of hTCM. After 2 weeks of incubation at 37° C. aliquots of wells containing proliferating cells were analysed with regard to their GPC3 specificity in an IFN-γ release assay and by multimer staining as described above. GPC3-specific CD8+ GPC3 MHC-multimer+ T cells were further expanded by restimulation with the feeder mixture as described.

### 1.7 IFN-γ Release Assay

T2 cells, which were used as targets, were loaded with 5 μM GPC3 peptide for 1.5 h prior to co-incubation with T cells. 2×10³ T cells were co-incubated with 1×10⁴ peptide loaded T2 cells in round-bottom 96-well plates yielding in an effector to target ratio of 0.2:1. T cells co-incubated with mock-peptide loaded T2 cells served as negative controls. After 24 h of co-culture supernatants were harvested and investigated by standard anti-human IFN-γ ELISA (BioLegend).

### 1.8 T Cell Receptor (TCR) Analysis and Cloning of Reactive TCR

For TCR analysis of GPC3 specific T cell clones RNA was extracted from T cell clones with Trizol and cDNA was synthesized using superscript II reverse transcriptase (Invitrogen). Variable parts of α- and β-chains were amplified with degenerated primer (Table 1) that cover 99% of the TCR variable gene segment families followed by agarose gel purification and DNA sequencing (GATC).

TABLE 1

| | FORWARD | REVERSE |
|---|---|---|
| ALPHA | 5′human panVa (“VPANHUM”): | 3′human CA2: |
| | 5′-TGAGTGTCCCPGAPGG2P-3′ | 5′-GTGACACATTTGTTTGAGAATC-3′ |
| | (P = A or G; 2 = A, G or T) | [SEQ ID NO. 43] |
| | [SEQ ID NO. 42] | |
| BETA | VP1: GCIITKTIYTGGTAYMGACA | CP1: |
| | [SEQ ID NO. 44] | GCACCTCCTTCCCATTCAC |
| | or | [SEQ ID NO. 46] |
| | VP2: CTITKTWTTGTAYCIKCAG | |
| | (I = inosine, W = A/T, M = A/C, | |
| | Y = C/T, K = G/T) | |
| | [SEQ ID NO. 45] | |

Variable parts of α and β chains of a GPC3 specific TCR were codon optimized for improved gene expression (Geneart) and cloned into the retroviral vector pMP71 via EcoRI and NotI restriction sites. Constant chains were murinized to improve pairing of the transgenic TCR and α- and β-chain were linked via a P2A element.

1.9 Retroviral TCR Transfer into Human PBMC

293T cells were co-transfected with 2 μg retroviral plasmid pMP71-GPC3-TCR, 1 μg pcDNA3.1-Mo-MLV and 1 μg pALF-10A1 the later representing plasmids that carry the retroviral genes for gag/pol and env, respectively. Transfection was performed with 10 lipofectamine according to the standard lipofectamin transfection protocol. Freshly isolated human PBMCs were activated on anti-CD28 and OKT3 coated 24-well plates in hTCM supplemented with 300 IU/ml IL-2 for two days. Activated PBMCs were transduced twice, on day 3 and 4. Virus containing supernatant of transfected 293T cells was harvested two days after transfection and added to RetroNectin-coated 24-well culture plates. The virus was spinoculated for 2 h at 32° C. and 2000 g. After centrifugation media was replaced with 1×10⁶ activated T cells/well in hTCM supplemented with 100 IU/ml IL-2. T cells were spinoculated with 1000 g for 10 min at 32° C. After 24 h of incubation medium was replaced by fresh medium containing 50 IU/ml IL2 and T cells were incubated for additional 48 h. Transduced PBMC were analysed by GPC3 MHC-multimer staining and in functional assay at multiple time points.

### 1.10 Functional Assays

Transduced PBMC were analysed with regard to their IFN-γ secretion capacity after co-incubation with different target cells as described above. Peptide concentrations and effector to target ratios varied. To assess killing ability of transduced PBMC the cells were co-incubated with HepG2 cells that were seeded on E-plate 96 culture plates (Roche). Target cell viability was measured on the XCelligence (Roche) over time. Alternatively cell titre blue assay was performed after 24 h of co-incubation.

### 2. Results

### 2.1 Identification of Immunodominant GPC3 Peptides Presented on MHC Class I Molecules of Human Hepatoma Cells

Immunodominant epitopes for GPC3 have not been described yet. In this study we used Ultra Nano HPLC coupled on-line to the Q Exactive mass spectrometer to obtain a comprehensive HLA class I peptidome from a GPC3 and HLA-A2 positive hepatoma cell line. The resulting data were analysed using the MaxQuant bioinformatics platform. Two HLA-A2 bound GPC3 peptides could be identified, herein referred to as
GPC3 peptide 367 or GPC3₃₆₇ or GPC3-P1:

[SEQ ID NO. 14]
FIDKKVLKV

and
GPC3 peptide 326 or GPC3₃₂₆ or GPC3-P2: (described in Komori et al., 2006)

[SEQ ID NO. 16]
TIHDSIQYV.

The knowledge of these two peptides enables us to target GPC3 epitopes that are presented on GPC3 positive HCC cells.

2.2 In Vitro Production of Glypican-3 mRNA and Expression of GPC3 in DCs after mRNA Electroporation

A construct containing GPC3 has been cloned, from which GPC3 mRNA can be produced. Glypican-3 expression has been shown from the plasmid pcDNA3.1(−)GPC3 after transfection of 293T cells. Expression of GPC3 from this construct after electroporation of DCs was optimized. We were able to detect GPC3 after electroporation of DCs. GPC3 was also expressed when co-electroporated with HLA-A2. HLA-A2 expression was stable for at least 24 h. In contrast GPC3 could only be detected up to 2 h post electroporation and was rapidly degraded thereafter.

### 2.3 Allorestricted Stimulation of T Cells

To isolate tumour reactive high avidity T cells, an allo-restricted stimulation approach (see FIG. 3) was used, as described by Wilde et. al., 2012. For stimulation of naïve T cells, autologous dendritic cells were co-transfected with GPC3 and HLA-A2 RNA and used as antigen presenting cells. T cells from the naïve T cell repertoire of HLA-A2 negative donors were co-cultured with and expanded on these HLA-A2+ GPC3+ DCs. After two weeks, MHC streptamer-positive CD8+ T cells specific for both targeted GPC3 epitopes were detected (<1%). We were able to enrich these cell populations further to 57% GPC3-P1- and 35% GPC3-P2-MHC streptamer-positive T cell lines and grew T cell clones from them. In a co-culture with GPC3-P1/-P2 peptide loaded T2 cells we identified T cell clones displaying specific effector function by IFNγ secretion (see FIG. 4). The specific effector function could be verified in an intracellular cytokine staining for IFNγ and TNFα. Furthermore T cell clones secreted IFNγ when co-cultured with HLA-A2+ GPC3+ hepatoma cells (HepG2). Functional T cell clones showed strong GPC3 MHC streptamer binding.

### 2.4 T Cells Specific for Peptide GPC3₃₆₇

We have identified and cloned the T cell receptor (TCR) specific for GPC3 peptide 367 from our T cell clones. This TCR is also referred to as P1-1. The T cell receptor sequence coding for the variable parts of alpha and beta chain of P1-1 were codon optimized to increase the level of expression after viral transfer into T cells. Furthermore the constant domains were exchanged with murine constant domains to avoid miss pairing with endogenous TCR chains and hence ensure optimal expression (see FIG. 5).

T cells engrafted with this optimized GPC3 specific TCR showed strong GPC3 MHC streptamer binding. When co-cultured with GPC3 peptide loaded target cells or a GPC3 expressing hepatoma cell line (HepG2), GPC3 TCR transduced T cells secreted IFNγ, TNFα, IL2 and the degranulation marker Lamp-1 (see FIG. 6A and data not shown). Furthermore cytotoxicity was observed by killing of up to 60% of HepG2 cells (see FIG. 6B).

The features disclosed in the foregoing description, in the claims and/or in the accompanying drawings may, both separately and in any combination thereof, be material for realizing the invention in diverse forms thereof.

### REFERENCES

Baumhoer D, Tornillo L, Stadlmann S, Roncalli M, Diamantis E K, Terracciano L M. Glypican 3 expression in human nonneoplastic, preneoplastic, and neoplastic tissues: a tissue microarray analysis of 4,387 tissue samples. Am J Clin Pathol. 2008 June; 129(6):899-906.
Boulter J M and Jakobsen B K. Stable, soluble, high-affinity, engineered T cell receptors: novel antibody-like proteins for specific targeting of peptide antigens. Clin Exp Immunol. 2005; 142(3): 454-460.
Csoka M, Strauss G, Debatin K M, Moldenhauer G. Activation of T cell cytotoxicity against autologous common acute lymphoblastic leukemia (CALL) blasts by CD3×CD19 bispecific antibody. Leukemia 1996 November; 10(11): 1765-72.
Engels B, Cam H, Schüler T, Indraccolo S, Gladow M, Baum C, Blankenstein T, Uckert W. Retroviral vectors for high-level transgene expression in T lymphocytes. Hum Gene Ther. 2003 Aug. 10; 14(12):1155-68.
Hamid O et al. Safety and tumor responses with lambrolizumab (anti-PD-1) in melanoma. N Engl J Med. 2013 Jul. 11; 369(2):134-44.
Han E Q, Li X L, Wang C R, Li T F, Han S Y. Chimeric antigen receptor-engineered T cells for cancer immunotherapy: progress and challenges. J Hematol Oncol. 2013 Jul. 8; 6:47.
Knabel, M., Franz, T. J., Schiemann, M., Wulf, A., Villmow, B., Schmidt., B., Bernhard, H., Wagner, H. and Busch, D. Reversible MHC multimer staining for functional isolation of T-cell populations and effective adoptive transfer. Nature Medicine 2002, 8 (6), 631-637.
Komori H, Nakatsura T, Senju S, Yoshitake Y, Motomura Y, Ikuta Y, Fukuma D, Yokomine K, Harao M, Beppu T, Matsui M, Torigoe T, Sato N, Baba H, Nishimura Y. Identification of HLA-A2- or HLA-A24-restricted CTL epitopes possibly useful for glypican-3-specific immunotherapy of hepatocellular carcinoma. Clin Cancer Res. 2006 May 1; 12(9):2689-97.
Lunde E, Løset GÅ, Bogen B, Sandlie I. Stabilizing mutations increase secretion of functional soluble TCR-Ig fusion proteins. BMC Biotechnology 2010, 10:61
Maiti S N, Huls H, Singh H, Dawson M, Figliola M, Olivares S, Rao P, Zhao Y J, Multani A, Yang G, Zhang L, Crossland D, Ang S, Torikai H, Rabinovich B, Lee D A, Kebriaei P, Hackett P, Champlin R E, Cooper L J. Sleeping beauty system to redirect T-cell specificity for human applications. J Immunother. 2013 February; 36(2): 112-23.
Miyoshi H, Blömer U, Takahashi M, Gage F H, Verma I M. Development of a self-inactivating lentivirus vector. J Virol. 1998 October; 72(10):8150-7.
Yu S F, von Rüden T, Kantoff P W, Garber C, Seiberg M, Rüther U, Anderson W F, Wagner E F, Gilboa E. Self-inactivating retroviral vectors designed for transfer of whole genes into mammalian cells. Prot Natl Acad Sci USA 1986 May; 83(10):3194-8.
Wilde S, Geiger C, Milosevic S, Mosetter B, Eichenlaub S, Schendel D J. Generation of allo-restricted peptide-specific T cells using RNA-pulsed dendritic cells: A three phase experimental procedure. Oncoimmunology. 2012 Mar. 1; 1 (2): 129-140.

1. A T-cell receptor (TCR) comprising:
(i) a T cell receptor α-chain comprising the amino acid sequence of SEQ ID NO. 1, or an amino acid sequence that has at least 80% identity to the amino acid sequence of SEQ ID NO. 1, or any variation of SEQ ID NO. 1 provided that said variation retains its functional ability to bind to the epitope with the amino acid sequence of SEQ ID NO. 14 or to its HLA-A2 bound form,
(ii) a T-cell receptor β-chain comprising the amino acid sequence of SEQ ID NO. 2, or an amino acid sequence that has at least 80% identity to the amino acid sequence of SEQ ID NO. 2, or any variation of SEQ ID NO. 2 provided that said variation retains its functional ability to bind to the epitope with the amino acid sequence of SEQ ID NO. 14 or to its HLA-A2 bound form, or
(iii) both (i) and (ii).

2. The T-cell receptor (TCR) of claim 1, comprising
(i) a T cell receptor α-chain further comprising the amino acid sequence of SEQ ID NO. 3, or an amino acid sequence that has at least 80% identity to the amino acid sequence of SEQ ID NO. 3,
(ii) a T-cell receptor β-chain further comprising the amino acid sequence of SEQ ID NO. 4, or an amino acid sequence that has at least 80% identity to the amino acid sequence of SEQ ID NO. 4,
(iii) a linker or hinge region comprising the amino acid sequence of SEQ ID NO. 5, or an amino acid sequence that has at least 80% identity to the amino acid sequence of SEQ ID NO. 5, or
(iv) any combination of (i), (ii), and (iii).

3. The T-cell receptor (TCR) of claim 1 comprising
(i) a T cell receptor α-chain comprising the amino acid sequence of SEQ ID NO. 6, or an amino acid sequence that has at least 80% identity to the amino acid sequence of SEQ ID NO. 6, or any variation of SEQ ID NO. 6 provided that said variation retains its functional ability to bind to the epitope with the amino acid sequence of SEQ ID NO. 14 or to its HLA-A2 bound form,
(ii) a T-cell receptor β-chain comprising the amino acid sequence of SEQ ID NO. 7, or an amino acid sequence that has at least 80% identity to the amino acid sequence of SEQ ID NO. 7, or any variation of SEQ ID NO. 7 provided that said variation retains its functional ability to bind to the epitope with the amino acid sequence of SEQ ID NO. 14 or to its HLA-A2 bound; form,
(iii) both (i) and (ii), or
(iv) the amino acid sequence of SEQ ID NO. 8.

4. A soluble T-cell receptor (sTCR) construct comprising
(1) at least one of
one or more T-cell receptor α-chains as defined in claim 1, and
one or more T-cell receptor β-chains as defined in claim 1,
(2) one or more fusion components selected from the group consisting of Fc receptors, Fc domains, cytokines, toxins, antibodies, and combinations thereof,
wherein the at least one T-cell receptor chain (1) is bound to the fusion component(s) (2), and
(3) a label.

5. A chimeric T-cell receptor comprising at least one of
the T-cell receptor α-chain as defined in claim 1,
the T-cell receptor β-chain as defined in claim 1, or
the T-cell receptor of claim 1,
wherein the TCR α-chain and/or the TCR β-chain is/are fused to CD3-zeta chain(s) and/or other TCR stimulation domains.

6. A bi-specific antibody comprising
(a) the T-cell receptor α- and β-chain(s) of the TCR as defined in claim 1, which are linked with each other and fused to
(b) an antibody or a single chain antibody fragment (scFv) which is directed against an antigen or epitope on the surface of lymphocytes.

7. A nucleic acid encoding the T-cell receptor according to claim 1.

8. A nucleic acid comprising
(i) the nucleic acid encoding for the amino acid sequence of SEQ ID NO. 1 and/or SEQ ID NO. 2, or the nucleotide sequence of SEQ ID NO. 9 and/or SEQ ID NO. 10 or their complementary sequence(s), or sequence(s) that have at least 80% identity to the nucleotide sequence of SEQ ID NO. 9 or 10;
(ii) the nucleic acid encoding for the amino acid sequence of SEQ ID NO. 6 and/or SEQ ID NO. 7, or the nucleotide sequence of SEQ ID NO. 11 and/or SEQ ID NO. 12, or their complementary sequence(s), or sequence(s) that have at least 80% identity to the nucleotide sequence of SEQ ID NO. 11 or 12; or
(iii) the nucleic acid encoding for the amino acid sequence of SEQ ID NO. 8, or the nucleotide sequence of SEQ ID NO. 13, or their complementary sequence(s), or sequence(s) that have at least 80% identity to the nucleotide sequence of SEQ ID NO. 13.

9. An expression construct for expressing the T-cell receptor according to claim 1 in a cell.

10. A cell comprising the expression construct of claim 9, wherein the cell is a lymphocyte.

11. A pharmaceutical composition comprising
the T-cell receptor of claim 1, and;
one or more pharmaceutically acceptable excipients.

12. A method of generating genetically modified lymphocytes, comprising
providing lymphocytes,
providing the T-cell receptor of claim 1,
introducing the T-cell receptor into the lymphocytes, thereby obtaining genetically modified lymphocytes.

13. (canceled)

14. (canceled)

15. (canceled)

16. A method of preventing and/or treating hepatocellular carcinoma, or cancers expressing GPC3, comprising the steps of
(a) providing lymphocytes of a patient or a blood donor;
(b) providing
the T-cell receptor of claim 1;
(c) ex vivo introduction of the T-cell receptor into the lymphocytes of step (a) and, thereby, obtaining genetically modified lymphocytes,
(d) administering the genetically modified lymphocytes of step (c) to a subject or patient in need thereof,
wherein the subject or patient is HLA-A2 positive.

17. A method of preventing and/or treating hepatocellular carcinoma, or cancers expressing GPC3,
comprising the steps of
(a) providing
the T-cell receptor of claim 1, and
(b) direct application, via injection or infusion, of the T-cell receptor of (a) to a subject or patient in need thereof,
wherein the subject or patient is HLA-A2-positive.

18. A method of detecting, diagnosing, prognosing,
preventing and/or treating hepatocellular carcinoma or cancers expressing GPC3, comprising the detection and/or destruction of cancer cells of a patient with the use of the soluble T-cell receptor construct of claim 4, wherein the soluble T-cell receptor is applied by intravenous, subcutaneous or intramuscular infusion or injection, or by application to the mucosa of the respiratory tract by inhalation or by spraying, wherein the patient is HLA-A2-positive.

19. A method for detecting hepatocellular carcinoma, or cancers expressing GPC3, comprising the in vitro staining of cancer cells by using the soluble T-cell receptor construct of claim 4 which is labeled.

20. A composition comprising a peptide comprising at least one GPC3 epitope comprising the amino acid sequence of SEQ ID NO. 14.

21. A nucleic acid molecule coding for at least one peptide according to claim 20 or a plasmid comprising at least one such nucleic acid molecule.

22. (canceled)

23. (canceled)

24. (canceled)

25. A vaccine composition comprising
(i) at least one peptide according to claim 20, and
(ii) an excipient.

26. (canceled)

27. A method of preventing and/or treating hepatocellular carcinoma or cancers expressing GPC3, comprising the step of administering a peptide of claim 20
to a subject in need thereof.
